SEQUENCE LISTING (1 sequence)

SEQ ID NO: 1 - DNA, 39 bp, Artificial Sequence
  Synthetic c5 specific aptamer
  misc_feature (1)..(1): May be 2′-fluoro-2′-deoxycytidine
  misc_feature (2)..(2): May be 2′-O-Methyl-2′-deoxyguanosine
  misc_feature (3)..(3): May be 2′-fluoro-2′-deoxycytidine
  misc_feature (4)..(4): May be 2′-fluoro-2′-deoxycytidine
  misc_feature (5)..(5): May be 2′OH-guanosine
  misc_feature (6)..(6): May be 2′-fluoro-2′-deoxycytidine
  misc_feature (7)..(7): May be 2′-O-Methyl-2′-deoxyguanosine
  misc_feature (8)..(8): May be 2′-O-Methyl-2′-deoxyguanosine
  misc_feature (9)..(9): May be 2′-fluoro-2′-deoxyuridine
  misc_feature (10)..(10): May be 2′-fluoro-2′-deoxycytidine
  misc_feature (11)..(11): May be 2′-fluoro-2′-deoxyuridine
  misc_feature (12)..(12): May be 2′-fluoro-2′-deoxycytidine
  misc_feature (13)..(13): May be 2′-O-Methyl-2′-deoxyadenosine
  misc_feature (14)..(14): May be 2′-O-Methyl-2′-deoxyguanosine
  misc_feature (15)..(lS): May be 2′-O-Methyl-2′-deoxyguanosine
  misc_feature (16)..(16): May be 2′-fluoro-2′-deoxycytidine
  misc_feature (17)..(17): May be 2′OH-guanosine
  misc_feature (18)..(18): May be 2′-fluoro-2′-deoxycytidine
  misc_feature (19)..(19): May be 2′-fluoro-2′-deoxyuridine
  misc_feature (20)..(20): May be 2′-O-Methyl-2′-deoxyguanosine
  misc_feature (21)..(21): May be 2′-O-Methyl-2′-deoxyadenosine
  misc_feature (22)..(22): May be 2′-O-Methyl-2′-deoxyguanosine
  misc_feature (23)..(23): May be 2′-fluoro-2′-deoxyuridine
  misc_feature (24)..(24): May be 2′-fluoro-2′-deoxycytidine
  misc_feature (25)..(25): May be 2′-fluoro-2′-deoxyuridine
  misc_feature (26)..(26): May be 2′-O-Methyl-2′-deoxyguanosine
  misc_feature (27)..(27): May be 2′-O-Methyl-2′-deoxyadenosine
  misc_feature (28)..(28): May be 2′-O-Methyl-2′-deoxyguanosine
  misc_feature (29)..(29): May be 2′-fluoro-2′-deoxyuridine
  misc_feature (30)..(30): May be 2′-fluoro-2′-deoxyuridine
  misc_feature (31)..(31): May be 2′-fluoro-2′-deoxyuridine
  misc_feature (32)..(32): May be 2′OH-adenosine
  misc_feature (33)..(33): May be 2′-fluoro-2′-deoxycytidine
  misc_feature (34)..(34): May be 2′-fluoro-2′-deoxycytidine
  misc_feature (35)..(35): May be 2′-fluoro-2′-deoxyuridine
  misc_feature (36)..(36): May be 2′-O-Methyl-2′-deoxyguanosine
  misc_feature (37)..(37): May be 2′-fluoro-2′-deoxycytidine
  misc_feature (38)..(38): May be 2′-O-Methyl-2′-deoxyguanosine
  misc_feature (39)..(39): May be an inverted orientation T (3′-3′-linked)
cgccgcggucucaggcgcugagucugaguuuaccugcgt

(19) United States
(12) Patent Application Publication
(10) Pub. No.: US 2023/0398062 A1
(43) Pub. Date: Dec. 14, 2023

(54) METHODS FOR TREATING OPHTHALMOLOGICAL CONDITIONS

(71) Applicant: IVERIC bio, Inc., Parsippany, NJ (US)

(72) Inventors: Pravin Dugel, Paradise Valley, AZ (US); Dhaval B. Desai, Cinnaminson, NJ (US)

(73) Assignee: IVERIC bio, Inc., Parsippany, NJ (US)

(21) Appl. No.: 18/034,821

(22) PCT Filed: Oct. 31, 2021
(86) PCT No.: PCT/US2021/057484

§ 371 Date: May 1, 2023

Related U.S. Application Data
(60) Provisional application No. 63/108,428, filed on Nov. 1, 2020.
(60) Provisional application No. 63/108,427, filed on Nov. 1, 2020.
(60) Provisional application No. 63/212,098, filed on Jun. 17, 2021.
(60) Provisional application No. 63/212,102, filed on Jun. 17, 2021.
(60) Provisional application No. 63/257,565, filed on Oct. 19, 2021.

(51) Int. Cl. A61K 9/00; A61P 27/02; A61K 31/7115

(52) U.S. Cl. CPC A61K 9/0048; A61P 27/02; A61K 31/7115

(57) ABSTRACT

The present invention relates to methods for treating a subject with risk factors for progression to iRORA, iRORA, nGA, cRORA, GA, AMD, intermediate AMD, dry AMD, and/or wet AMD, in a subject in need thereof comprising administration of an effective amount of an anti-C 5 agent or a pharmaceutically acceptable salt thereof. The present invention also relates to methods for treating an ophthalmological disease, disorder, and/or condition in a subject with high-risk drusen, comprising administration of an effective amount of an anti-C 5 agent or a pharmaceutically acceptable salt thereof.

### CROSS-REFERENCE TO RELATED APPLICATION(S)

This application claims the benefit of: U.S. Provisional Patent Application No. 63/108,427, which was filed on Nov. 1, 2020; U.S. Provisional Patent Application No. 63/108,428, which was filed on Nov. 1, 2020; U.S. Provisional Patent Application No. 63/212,102, which was filed on Jun. 17, 2021; and U.S. Provisional Patent Application No. 63/212,098, which was filed on Jun. 17, 2021, and U.S. Provisional Patent Application No. 63/257,565, all of which are incorporated by reference in their entireties.

### SEQUENCE LISTING

The Sequence Listing associated with this application is provided in text format in lieu of a paper copy, and is hereby incorporated by reference into the specification. The name of the text file containing the Sequence Listing is seq.txt. The txt file is about 4 KB, was created on 1 Nov. 2020, and is being submitted electronically via EFS-Web.

### FIELD OF THE INVENTION

The present invention relates to methods for treating subjects with risk factors for progression to iRORA, iRORA, nGA, cRORA, GA, AMD, intermediate AMD, dry AMD, and/or wet AMD, including in a subject with high-risk drusen comprising administering an effective amount of an anti-C5 agent or a pharmaceutically acceptable salt thereof.

### BACKGROUND OF THE INVENTION

Age-related macular degeneration (“AMD”) is a disease characterized by progressive degenerative abnormalities in the macula, a region in the central portion of the retina. Age-related macular degeneration is a complex, gradually progressing disorder of the eye that leads to distortions and/or blind spots (scotoma), changes in dark adaptation (diagnostic of rod cell health), changes in color interpretation (diagnostic of cone cell health), a decrease in visual acuity, or irreversible blindness.

AMD is typically a disease of the elderly and is the leading cause of blindness in individuals >50 years of age in developed countries. In the United States, it is estimated that approximately 6% of individuals 65-74 years of age, and 20% of those older than 75 years of age, are affected with AMD. Because of increasing life expectancy in developed and developing countries, the elderly portion of the general population is expected to increase at the greatest rate in coming decades. In the absence of adequate prevention or treatment measures, the number of cases of AMD with visual loss is expected to grow in parallel with the aging population.

Non-exudative AMD is the non-neovascular (“dry”) form of the disease (“dry AMD”) Dry AMD accounts for approximately 90% of all AMD cases. Dry AMD can be characterized by degeneration of the macula and, with continued progression over multiple years, may ultimately result in atrophy of the central retina associated with central vision loss, also known as geographic atrophy. Dry AMD is a significant cause of moderate and severe loss of central vision and is bilateral in most patients. In dry AMD, thinning of the retinal pigment epithelial cells (RPE) in the macula develops, along with other age-related changes to the adjacent retinal tissue layers.

Choroidal neovascularization can be an early sign of wet AMD. Once neovascularization arises in non-exudative AMD and begins to leak, the disease is referred to as exudative AMD, the neovascular (“wet”) form of the disease (“wet AMD”), with non-exudative AMD still present and potentially progressing in the patient. Wet AMD may cause sudden, often substantial, loss of central vision, particularly if untreated.

Recent advancements in imaging technology, specifically optical coherence tomography (“OCT”), and more specifically spectral domain optical coherence tomography (“SD-OCT”), have led to an ability to reproducibly and reliably measure morphological changes in the eyes of subjects exhibiting AMD disease states and to monitor the progression of disease over time. Such disease states include incomplete retinal pigment epithelium (“RPE”) and outer retinal atrophy (“iRORA”), risk factors for the progression to iRORA, intermediate AMD, complete RPE and outer retinal atrophy (“cRORA”), nascent geographic atrophy (“nGA”), and/or geographic atrophy (“GA”). See e.g., Guymer et al., Incomplete Retinal Pigment Epithelial and Outer Retinal Atrophy in Age-Related Macular Degeneration: Classification of Atrophy Meeting Report 4, Ophthalmology 2020; 127:394-409; see also Wu et al., Optical Coherence Tomography-Defined Changes Preceding the Development of Drusen-Associated Atrophv in Age-Related Macular Degeneration, Ophthalmology 2014:121:2415-2422.

Drusen are focal accumulations of extracellular material (including lipids and proteins), and typically are located between the basal lamina of the RPE and the inner collagenous layer of Bruch's membrane. Drusen are thought to be one of the earliest signs of AMD. Drusen are dynamic structures that can increase in size, coalesce, or regress and may vary in number, shape, and distribution. Drusen can go through repeated cycles of growth and shrinkage. Certain morphological characteristics of drusen have been associated with risk for progression to late-AMD, including GA, macular neovascularization, or both.

Studies measuring the natural history of drusen morphology have identified three different drusen growth patterns. See Filho et al., Change in Drusen Volume as a Novel Clinical Trial Endpoint for the Study of Complement Inhibition in Age-related Macular Degeneration, Ophthalmic Surg Lasers Imaging Retina. 2014,45:18-31 at 18. For example, it was observed that most eyes showed an increase in drusen volume and some eyes showed stable drusen morphology. While some eyes showed a decrease in drusen volume without obvious sequelae, this was seen to be a “rare event.” Id. Furthermore, it is thought that the pathologic processes responsible for disease progression in different stages of disease may be different. E.g. Schaal et al., Anatomic Clinical Trial Endpoints for Nonexudative Age-Related Macular Degeneration, Ophthalmology 2016, 123:1060-1079 at 1075. Consequently, an effective therapy for slowing development of GA in late AMD, for example, may or may not be beneficial if applied to an earlier disease state. Id

Therefore, there is a need for therapies for treating patients exhibiting risk factors for progression to iRORA, iRORA, nGA, cRORA, GA, AMD, intermediate AMD, dry AMD, and/or wet AMD, including in a subject with high-risk drusen.

### SUMMARY OF THE INVENTION

Aspects of the present invention relate to methods of treating a subject having risk factors for the progression to iRORA.

Aspects of the present invention relate to methods of treating a subject having iRORA.

Aspects of the present invention relate to methods of treating a subject having nGA.

Aspects of the present invention relate to methods of treating a subject having cRORA.

Aspects of the present invention relate to methods of treating a subject having early-stage AMD.

Aspects of the present invention relate to methods of treating a subject having intermediate AMD.

Aspects of the present invention relate to methods useful for the treatment of an ophthalmological disease or disorder in a subject with high-risk drusen.

Aspects of the present invention relate to methods of treating a subject having high-risk drusen.

Aspects of the present invention relate to methods of treating a subject having high-risk drusen and risk factors for the progression to iRORA.

Aspects of the present invention relate to methods of treating a subject having high-risk drusen and risk factors for the progression to cRORA.

Aspects of the present invention relate to methods of treating a subject having high-risk drusen and iRORA.

Aspects of the present invention relate to methods of treating a subject having high-risk drusen and nGA.

Aspects of the present invention relate to methods of treating a subject having high-risk drusen and cRORA.

Aspects of the present invention relate to methods of treating a subject having high-risk drusen and GA.

Aspects of the present invention relate to methods of treating a subject having high-risk drusen and AMD.

Aspects of the present invention relate to methods of treating a subject having high-risk drusen and intermediate AMD.

Aspects of the present invention relate to methods of treating a subject having high-risk drusen and dry AMD.

Aspects of the present invention relate to methods of treating a subject having high-risk drusen and wet AMD.

Aspects of the present invention relate to methods of treating a subject having drusen that are <63 μm in diameter.

Aspects of the present invention relate to methods of treating a subject having drusen between ≥63 μm and <125 μm in diameter.

Aspects of the present invention relate to methods of treating a subject having drusen of ≥125 μm in diameter.

Aspects of the present invention relate to methods of treating a subject having drusen that are less than 63 μm in diameter.

Aspects of the present invention relate to methods of treating a subject having drusen between greater than or equal to 63 μm and less than 125 μm in diameter.

Aspects of the present invention relate to methods of treating a subject having drusen of greater than or equal to 125 μm in diameter.

Aspects of the present invention relate to methods of treating a subject having drusen that are about <63 μm in diameter.

Aspects of the present invention relate to methods of treating a subject having drusen between about >63 μm and about <125 μm in diameter.

Aspects of the present invention relate to methods of treating a subject having drusen of about ≥125 μm in diameter.

Aspects of the present invention relate to methods of treating a subject having high risk drusen and early-stage, intermediate, or late-stage AMD.

Aspects of the present invention relate to methods of treating an ophthalmological disease, disorder, and/or condition comprising administering an effective amount of an anti-C5 agent or a pharmaceutically acceptable salt thereof to a subject in need thereof having: (i) high-risk drusen and (ii) an ophthalmological disease, disorder, and/or condition selected from the group consisting of risk factors for the progression to iRORA, iRORA, nGA, cRORA, GA, AMD, intermediate AMD, dry AMD, and wet AMD.

Aspects of the present invention relate to methods of treating an ophthalmological disease, disorder, and/or condition comprising administering an effective amount of an anti-C5 agent or a pharmaceutically acceptable salt thereof to a subject in need thereof having: (i) high-risk drusen and (ii) an ophthalmological disease, disorder, and/or condition selected from the group consisting of risk factors for the progression to iRORA, iRORA, nGA, cRORA, GA, AMD, intermediate AMD, dry AMD, and wet AMD, wherein the progression of the ophthalmological disease, disorder, and/or condition is reduced, slowed, inhibited, prevented, improved, and/or reversed.

Aspects of the present invention relate to methods of treating an ophthalmological disease, disorder, and/or condition comprising administering an effective amount of an anti-C5 agent or a pharmaceutically acceptable salt thereof to a subject in need thereof having: (i) high-risk drusen and (ii) an ophthalmological disease, disorder, and/or condition selected from the group consisting of risk factors for the progression to iRORA, iRORA, nGA, cRORA, GA, AMD, intermediate AMD, dry AMD, and wet AMD, wherein at least one characteristic of drusen (e.g., number, extent, area, volume, shape, density, and reflectivity) is reduced, slowed, inhibited, prevented, improved and/or reversed.

Aspects of the present invention relate to methods of treating an ophthalmological disease, disorder, and/or condition comprising administering an effective amount of an anti-C5 agent or a pharmaceutically acceptable salt thereof to a subject in need thereof having: (i) high-risk drusen and (ii) an ophthalmological disease, disorder, and/or condition selected from the group consisting of risk factors for the progression to iRORA, iRORA, nGA, cRORA, GA, AMD, intermediate AMD, dry AMD, and wet AMD, wherein the progression of the ophthalmological disease, disorder, and/or condition is reduced, slowed, inhibited, prevented, improved, and/or reversed, and wherein at least one characteristic of drusen (e.g., number, extent, area, volume, shape, density, and reflectivity) is reduced, slowed, inhibited, prevented, improved and/or reversed.

Aspects of the present invention relate to methods of treating an ophthalmological disease, disorder, and/or condition selected from the group consisting of risk factors for the progression to iRORA, iRORA, nGA, intermediate AMD, and cRORA, comprising administering to a subject in need thereof an effective amount of an anti-C5 agent or a pharmaceutically acceptable salt thereof.

Aspects of the present invention relate to methods of treating an ophthalmological disease, disorder, and/or condition selected from the group consisting of risk factors for the progression to iRORA, iRORA, nGA, cRORA, GA, AMD, intermediate AMD, dry AMD, and wet AMD, comprising administering to a subject in need thereof an effective amount of an anti-C5 agent or a pharmaceutically acceptable salt thereof, wherein the subject has high-risk drusen.

Aspects of the present invention relate to methods of treating an ophthalmological disease, disorder, and/or condition selected from the group consisting of risk factors for the progression to iRORA, iRORA, nGA, intermediate AMD, and cRORA, comprising administering to a subject in need thereof an effective amount of an anti-C5 agent or a pharmaceutically acceptable salt thereof, and wherein the progression of the ophthalmological disease, disorder, and/or condition is reduced, slowed, inhibited, prevented, improved, and/or reversed.

Aspects of the present invention relate to methods of treating an ophthalmological disease, disorder, and/or condition selected from the group consisting of risk factors for the progression to iRORA, iRORA, nGA, cRORA, GA, AMD, intermediate AMD, dry AMD, and wet AMD, comprising administering to a subject in need thereof an effective amount of an anti-C5 agent or a pharmaceutically acceptable salt thereof, wherein the subject has high-risk drusen, and wherein the progression of the ophthalmological disease, disorder, and/or condition is reduced, slowed, inhibited, prevented, improved, and/or reversed.

Aspects of the present invention relate to methods of treating an ophthalmological disease, disorder, and/or condition selected from the group consisting of risk factors for the progression to iRORA, iRORA, nGA, intermediate AMD, and cRORA, comprising administering to a subject in need thereof an effective amount of an anti-C5 agent or a pharmaceutically acceptable salt thereof, wherein at least one characteristic of drusen (e.g., number, extent, area, volume, shape, density, and reflectivity) is reduced, slowed, inhibited, prevented, improved and/or reversed.

Aspects of the present invention relate to methods of treating an ophthalmological disease, disorder, and/or condition selected from the group consisting of risk factors for the progression to iRORA, iRORA, nGA, cRORA, GA, AMD, intermediate AMD, dry AMD, and wet AMD, comprising administering to a subject in need thereof an effective amount of an anti-C5 agent or a pharmaceutically acceptable salt thereof, wherein the subject has high-risk drusen, wherein at least one characteristic of drusen (e.g., number, extent, area, volume, shape, density, and reflectivity) is reduced, slowed, inhibited, prevented, improved and/or reversed.

Aspects of the present invention relate to methods of treating an ophthalmological disease, disorder, and/or condition selected from the group consisting of risk factors for the progression to iRORA, iRORA, nGA, intermediate AMD, and cRORA, comprising administering to a subject in need thereof an effective amount of an anti-C5 agent or a pharmaceutically acceptable salt thereof, wherein the progression of the ophthalmological disease, disorder, and/or condition is reduced, slowed, inhibited, prevented, improved, and/or reversed, and wherein at least one characteristic of drusen (e.g., number, extent, area, volume, shape, density, and reflectivity) is reduced, slowed, inhibited, prevented, improved and/or reversed.

Aspects of the present invention relate to methods of treating an ophthalmological disease, disorder, and/or condition selected from the group consisting of risk factors for the progression to iRORA, iRORA, nGA, cRORA, GA, AMD, intermediate AMD, dry AMD, and wet AMD, comprising administering to a subject in need thereof an effective amount of an anti-C5 agent or a pharmaceutically acceptable salt thereof, wherein the subject has high-risk drusen, wherein the progression of the ophthalmological disease, disorder, and/or condition is reduced, slowed, inhibited, prevented, improved, and/or reversed, and wherein at least one characteristic of drusen (e.g., number, extent, area, volume, shape, density, and reflectivity) is reduced, slowed, inhibited, prevented, improved and/or reversed.

Aspects of the present invention relate to methods of treatment, wherein the anti-C5 agent comprises a C5-specific aptamer, in which the aptamer has a sequence of fCmGfCfCGfCmGmGfUJfCfUfCmAmGmGfCGfCfUmGmAmGfUfCfUmGmAmGffRJfAfCf CfUmGfCmG-3T (SEQ ID NO: 1), in which fC and fU=2′ fluoro nucleotides, mG and mA=2′-OMe nucleotides, all other nucleotides are 2′-OH, and 3T indicates an inverted deoxythymidine.

Aspects of the present invention relate to administering an anti-C5 agent with another agent, such an HTRA1 inhibitor compound or a VEGF antagonist.

### BRIEF DESCRIPTION OF THE DRAWINGS

FIG. 1 illustrates a bar graph comparing the progression from large drusen to iRORA or cRORA after administration of 2 mg of an anti-C5 agent versus a sham at 6 months, 12 months, and 18 months.

FIG. 2 illustrates the data in FIG. 1 in a line graph.

FIG. 3 illustrates a bar graph comparing the progression from iRORA to cRORA after administration of 2 mg of an anti-C5 agent versus a sham at 6 months, 12 months, and 18 months.

FIG. 4 illustrates the data in FIG. 3 in a line graph.

FIG. 5 illustrates a table comparing baseline characteristics in terms of GA lesion size, presence of large drusen, presence of iRORA, lesion location, and lesion focality for subjects administered 2 mg of an anti-C5 agent or sham.

FIG. 6 illustrates a graph comparing the least-squares mean change from baseline GA area after administration of 2 mg of an anti-C5 agent versus a sham at 6 months and 12 months.

FIG. 7 illustrates a graph comparing the least-squares mean change from baseline in square-root GA area after administration of 2 mg of an anti-C5 agent versus a sham at 6 months, 12 months, and 18 months.

### DETAILED DESCRIPTION OF THE INVENTION

One aspect of the present invention relates to methods of treating an ophthalmological disease, disorder, and/or condition selected from the group consisting of risk factors for the progression to iRORA, iRORA, nGA, intermediate AMD, and cRORA, comprising administering to a subject in need thereof an effective amount of an anti-C5 agent or a pharmaceutically acceptable salt thereof.

One aspect of the present invention relates to methods and compositions useful in treating a subject having: (i) high-risk drusen and (ii) an ophthalmological disease, disorder, and/or condition selected from the group consisting of risk factors for the progression to iRORA, iRORA, nGA, cRORA, GA, AMD, intermediate AMD, dry AMD, and wet AMD, comprising the administration of a pharmaceutically acceptable amount of an anti-C5 agent or a pharmaceutically acceptable salt thereof.

Another aspect of the present invention relates to methods of treating an ophthalmological disease, disorder, and/or condition selected from the group consisting of risk factors for the progression to iRORA, iRORA, nGA, cRORA, GA, AMD, intermediate AMD, dry AMD, and wet AMD, comprising administering to a subject in need thereof an effective amount of an anti-C5 agent or a pharmaceutically acceptable salt thereof, wherein the subject has high-risk drusen.

### Anti-C5 Agents

The term “anti-C5 agent” refers to an agent that reduces, or inhibits, either partially or fully, the activity or production of a C5 complement protein or a variant thereof. An anti-C5 agent may directly or indirectly reduce or inhibit the activity or production of a C5 complement protein or variant thereof. An anti-C5 agent may reduce or inhibit the conversion of C5 complement protein into its component polypeptides C5a and C5b. Anti-C5 agents may also reduce or inhibit the activity or production of C5a and/or C5b.

In embodiments of the present invention, the anti-C5 agent comprises a C5-specific aptamer, in which the aptamer comprises a nucleotide sequence of fCmGfCfCGfCmGmGfUfCfUfCmAmGmGfCGfCfUmGmAmGfUflCfUmGmAmGfUfUfUAfCf CfUmGfCmG-3T (SEQ ID NO: 1), in which fC and fU=2′ fluoro nucleotides, mG and mA=2′-OMe nucleotides, all other nucleotides are 2′-OH, and 3T indicates an inverted deoxythymidine.

In some embodiments, the aptamer may be conjugated to a polyethylene glycol moiety (PEG) via a linker. The PEG moiety may have a molecular weight greater than about 10 kDa, such as a molecular weight of about 20 kDa, or about 30 kDa, or about 40 kDa, or about 50 kDa, or about 60 kDa. In some embodiments, the PEG moiety is conjugated via a linker to the 5′ end of the aptamer. In certain embodiments, the PEG moiety conjugated to the 5′ end is a PEG moiety of about 40 kDa molecular weight. In particular embodiments, the about 40 kDa PEG moiety is a branched PEG moiety. The branched about 40 kDa PEG moiety may be, for example, 1,3-bis(mPEG-[about 20 kDa])-propyl-2-(4′-butamide), or 2,3-bis(mPEG-[about 20 kDa])-propyl-1-carbamoyl.

The term “about” when used in connection with a referenced numeric indication means the referenced numeric indication plus or minus up to 20% of that referenced numeric indication. For example, “about 100” means from 80 to 120 and “about six” means from 4.8 to 7.2. In one embodiment, values described herein may vary less than 20% of the referenced numeric indication, such as 10%, 5%, or less.

In certain embodiments, the aptamer is a compound, ARC187, having the structure

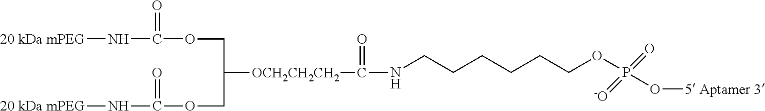

or a pharmaceutically acceptable salt thereof, where Aptamer=fCmGfCfCGfCmGmGfUfCfUfC mAmGmGfCGfCfJmGmAmGfUtCfUmGmAmGfUfUfUAfCfCfUmGfCmG-3T (SEQ ID NO: 1), in which fC and fU=2′-fluoro nucleotides, mG and mA=2′-OMe nucleotides, all other nucleotides are 2′-OH, and where 3T indicates an inverted deoxy thymidine. In some embodiments, each 20 kDa mPEG of the above structure has a molecular weight of about 20 kDa.

In certain embodiments, the aptamer is a compound, ARC1905, having the structure set forth below:

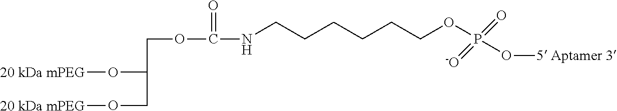

or a pharmaceutically acceptable salt thereof, where Aptamer=fCmGfCfCGfCmGmGfUfCfUfC mAmGmGfCGfCfUmGmAmGfUfCfUmGmAmGfUJfUUAfCfCfUmGfCmG-3T (SEQ ID NO: 1), in which fC and fU=2′-fluoro nucleotides, mG and mA=2′-OMe nucleotides, all other nucleotides are 2′-OH, and where 3T indicates an inverted deoxy thymidine. In some embodiments, each 20 kDa mPEG of the above structure has a molecular weight of about 20 kDa.

Examples of a pharmaceutically acceptable salt include, but are not limited to, sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, pamoate, phenylacetate, trifluoroacetate, acrylate, chlorobenzoate, dinitrobenzoate, hydroxybenzoate, methoxybenzoate, methylbenzoate, o-acetoxybenzoate, naphthalene-2-benzoate, isobutyrate, phenylbutyrate, α-hydroxybutyrate, butyne-1,4-dicarboxylate, hexyne-1,4-dicarboxylate, caprate, caprylate, cinnamate, glycollate, heptanoate, hippurate, malate, hydroxymaleate, malonate, mandelate, mesylate, nicotinate, phthalate, teraphthalate, propiolate, propionate, phenylpropionate, sebacate, suberate, p-bromobenzenesulfonate, chlorobenzenesulfonate, ethylsulfonate, 2-hydroxyethylsulfonate, methylsulfonate, naphthalene-1-sulfonate, naphthalene-2-sulfonate, naphthalene-1,5-sulfonate, xylenesulfonate, and tartarate salts. The term “pharmaceutically acceptable salt” includes, but is not limited to, a hydrate of a compound of the invention and also may refer to a salt of an antagonist of the present invention having an acidic functional group, such as, but not limited to, a carboxylic acid functional group or a hydrogen phosphate functional group, and a base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or tri-alkylamines, dicyclohexylamine; tributyl amine; pyridine; N-methyl, N-ethylamine; diethylamine; triethylamine; mono-, bis-, or tris-(2-OH-lower alkylamines), such as mono-, bis-, or tris-(2-hydroxyethyl)amine, 2-hydroxy-tert-butylamine, or tris-(hydroxymethyl)methylamine; N,N-di-lower alkyl-N-(hydroxyl-lower alkyl)-amines, such as N,N-dimethyl-N-(2-hydroxyethyl)amine or tri-(2-hydroxyethyl)amine; N-methyl-D-glucamine; and amino acids such as arginine, lysine, and the like.

### Early Stage AMD and Drusen

In certain aspects, the present invention relates to methods and compositions useful for subjects with incomplete retinal pigment epithelium (RPE) and outer retinal atrophy (“iRORA”). iRORA is an ophthalmological disease, disorder, and/or condition characterized by the following three features, which are vertically aligned and determined by OCT: (1) a region of signal hypertransmission into the choroid, (2) a corresponding zone of attenuation or disruption of the RPE, and (3) evidence or signs of overlying photoreceptor degeneration. Evidence or signs of overlying photoreceptor degeneration include subsidence of the inner nuclear layer (“INL”) and outer plexiform layer (“OPL”), presence of a hyporeflective wedge in the Henle fiber layer (“HFL”), thinning of the outer nuclear layer (“ONL”), disruption of the external limiting membrane (“ELM”), and disintegrity of the ellipsoid zone (“EZ”). iRORA should not be used to refer to an RPE tear. iRORA may progress to cRORA, nGA, GA, intermediate AMD, and/or wet AMD.

In certain aspects, the present invention relates to methods and compositions useful for subjects with risk factors for the progression to iRORA. A subject who exhibits risk factors for the progression to iRORA exhibits some, but not all, of the signs of iRORA, as described above. In addition, a subject exhibiting high-risk drusen and risk factors for the progression to iRORA may also exhibit hyperreflective foci, heterogeneous internal reflectivity of drusen, and/or subretinal drusenoid deposits. Determination of whether a subject has risk factors for the progression to iRORA is also accomplished with multimodal imaging, which includes but is not limited to OCT. A subject with risk factors for the progression to iRORA may progress to iRORA, cRORA, nGA, GA, intermediate AMD, and/or wet AMD.

In certain aspects, the present invention relates to methods and compositions useful for subjects with intermediate AMD. Intermediate AMD is a disease state that is between early stage AMD such as risk factors for progression to iRORA and later stage AMD such as cRORA. In some embodiments, intermediate AMD may include a subject exhibiting drusen, including high risk drusen, and including drusen with a diameter greater than 125 μm. In some embodiments, intermediate AMD may include a subject exhibiting drusen, including high risk drusen, and including drusen with a diameter greater than 125 μm, wherein the subject also exhibits iRORA. In some embodiments, intermediate AMD may include a subject exhibiting drusen, including high risk drusen, and including drusen with a diameter greater than 125 μm, wherein the subject also exhibits iRORA, and wherein the subject also exhibits hyperreflective foci.

In certain aspects, the present invention relates to methods and compositions useful for subjects with nGA. nGA is an ophthalmological disease, disorder, and/or condition characterized by: (i) the subsidence of the inner nuclear layer (INL) and outer plexiform later (OPL), and (ii) a hyporeflective wedge-shaped band within the OPL, including within the Henle fiber layer. nGA may also be accompanied by RPE disturbance and increased signal hypertransmission into the choroid. In addition, features frequently present in subsidence of the OPL and INL may include disruption of the inner segment ellipsoid (“ISe”), a break in the ELM, and traces of increased signal transmission below Bruch's membrane. Furthermore, features frequently present with a hyporeflective wedge-shaped band include a vortex-like subsidence of OPL and INL, drusen regression, and traces of increased signal transmission below RPE. The onset of nGA may also be accompanied by, or preceded by the regression of some or all drusen, resulting in the overlying retinal layers undergoing characteristic changes in progressive atrophy. nGA may also be associated with, and/or occur simultaneously with, an iRORA disease state. A subject exhibiting nGA may have previously exhibited risk factors for the progression to iRORA and may subsequently exhibit cRORA and/or GA.

In certain aspects, the present invention relates to methods and compositions useful for subjects with cRORA. cRORA is an ophthalmological disease, disorder, and/or condition meeting the requirements of iRORA and further requiring a change in the areas of RPE, hypertransmission having a diameter of at least 250 μm on the OCT B-scan, and evidence of photoreceptor loss. cRORA may progress to nGA, GA, and/or wet AMD.

In certain aspects, the present invention includes methods of administration to subjects with high-risk drusen. High-risk drusen refers to drusen associated with a high risk of AMD and/or a high risk of disease progression from an earlier-stage AMD to a later-stage AMD. High-risk drusen may have any of the following characteristics. For example, high-risk drusen may be characterized by the presence of at least one druse with a diameter of at least 250 μm observed on fundus biomicroscopy or color fundus photography and/or a total volume of drusen of at least 0.03 mm³ as measured by SD-OCT within a 3 mm diameter circle centered on the fovea. In some embodiments, high-risk drusen may have a diameter of at least 300 μm and exist within a 500 μm diameter circle centered on the fovea. In another embodiment, high-risk drusen is characterized by the presence of at least one druse with a diameter of at least about 150 μm.

In addition, high-risk drusen may be characterized by other morphological features. Furthermore, high-risk drusen may be characterized in terms of maximum lesion height and diameter, lesion internal reflectivity, presence and extent of overlying intraretinal hyperreflective foci, and choroidal thickness both subfoveally and below drusen. In addition, high-risk drusen may exhibit hyperreflective foci overlying the drusen, heterogeneous internal reflectivity of drusen, or choroidal thickness less than 135 μm below the drusen baseline. In addition, high-risk drusen may be soft, large, indistinct, and/or confluent.

Risk factors include, e.g., hypertension, obesity, atherosclerosis, focal deposition of acellular detritus between the RPE, family history of AMD, including a genetic risk, smoking, high body mass index, high-fat diet, low intake of antioxidants and zinc, previous cataract surgery, history of cardiovascular disease, higher plasma fibrinogen, and/or diabetes (see, e.g., Garcia-Layana et al., Clinical Interventions in Aging 2017:12 1579-1587, the contents of which are incorporated herein by reference in their entirety).

In certain aspects, the present invention relates to methods and compositions useful for subjects with high-risk drusen and incomplete RPE and iRORA. In certain aspects, the present invention relates to methods and compositions useful for subjects with high-risk drusen and risk factors for the progression to iRORA. In certain aspects, the present invention relates to methods and compositions useful for subjects with high-risk drusen and cRORA. In certain aspects, the present invention relates to methods and compositions useful for subjects with high-risk drusen and nGA.

In certain aspects, the present invention relates to methods and compositions useful for subjects with high-risk drusen and GA. When the condition is severe, dry AMD results in marked thinning and/or atrophy of the macula, resulting from the loss of the RPE and associated capillaries (choriocapillaris). This form of late stage dry AMD is associated with thinning and loss of function of the neural retinal located above the affected RPE. This collective phenotype in late stage dry AMD is termed GA. GA may also refer to a subset of cRORA, which may occur in the presence of or the absence of current or past macular neovascularization.

In certain aspects, the present invention relates to methods and compositions useful for subjects with drusen that are <63 μm in diameter (i.e., “drupelets”).

In certain aspects, the present invention relates to methods and compositions useful for subjects having drusen between ≥63 μm and <125 μm in diameter.

In certain aspects, the present invention relates to methods and compositions useful for subjects having drusen at ≥125 μm.

In certain aspects, the present invention relates to methods and compositions useful for subjects with high-risk drusen and exudative AMD, e.g., when new blood vessels invade the overlying retina (i.e. wet AMD).

In certain aspects, the present invention relates to methods and compositions useful for subjects with high-risk drusen and intermediate AMD.

In certain aspects, the subject exhibits hyperpigmentation or hypopigmentation. In some embodiments, increasing hyperpigmentation is another way to signify disease progression. In some embodiments, hypopigmentation is associated with specific disease states.

SD-OCT specifically provides a reliable and reproducible method for measuring drusen morphology over time as well as other characteristic features of AMD. Additionally, SD-OCT algorithms are available in order to quantify drusen characteristics. Such algorithms can be fully-automated and reliably report drusen load, drusen volume and area and morphological changes over time using cube root and square root transformations, respectively. Advancements of imaging with the use of SD-OCT and color fundus imaging has made it possible to study and measure the morphology of drusen by providing three-dimensional, geometric assessment. SD-OCT imaging has also allowed for multimodal imaging and has identified other macular features that increase the risk of vision loss, including decreased internal reflectivity of drusen (identified as calcified drusen), intraretinal hyperreflective foci, and subretinal drusenoid deposits. Instruments used for SD-OCT are known in the art, such as the Cirrus HD-OCT.

### Methods of Use

In one aspect, the present invention relates to methods of treating risk factors for the progression to iRORA comprising administering to a subject having risk factors for the progression to iRORA an effective amount of an anti-C5 agent or a pharmaceutically acceptable salt thereof.

In one aspect, the present invention relates to methods of treating iRORA comprising administering to a subject having iRORA an effective amount of an anti-C5 agent or a pharmaceutically acceptable salt thereof.

In one aspect, the present invention relates to methods of treating nGA comprising administering to a subject having nGA an effective amount of an anti-C5 agent or a pharmaceutically acceptable salt thereof.

In one aspect, the present invention relates to methods of treating intermediate AMD comprising administering to a subject having intermediate AMD an effective amount of an anti-C5 agent or a pharmaceutically acceptable salt thereof.

In one aspect, the present invention relates to methods of treating cRORA comprising administering to a subject having cRORA an effective amount of an anti-C5 agent or a pharmaceutically acceptable salt thereof.

In one aspect, the present invention relates to methods of treating risk factors for iRORA comprising administering to a subject having risk factors for iRORA an effective amount of an anti-C5 agent or a pharmaceutically acceptable salt thereof, wherein the progression of the ophthalmological disease, disorder, and/or condition is reduced, slowed, inhibited, prevented, improved, and/or reversed.

In one aspect, the present invention relates to methods of treating iRORA comprising administering to a subject having iRORA an effective amount of an anti-C5 agent or a pharmaceutically acceptable salt thereof, wherein the progression of the ophthalmological disease, disorder, and/or condition is reduced, slowed, inhibited, prevented, improved, and/or reversed.

In one aspect, the present invention relates to methods of treating nGA comprising administering to a subject having nGA an effective amount of an anti-C5 agent or a pharmaceutically acceptable salt thereof, wherein the progression of the ophthalmological disease, disorder, and/or condition is reduced, slowed, inhibited, prevented, improved, and/or reversed.

In one aspect, the present invention relates to methods of treating intermediate AMD comprising administering to a subject having intermediate AMD an effective amount of an anti-C5 agent or a pharmaceutically acceptable salt thereof, wherein the progression of the ophthalmological disease, disorder, and/or condition is reduced, slowed, inhibited, prevented, improved, and/or reversed.

In one aspect, the present invention relates to methods of treating cRORA comprising administering to a subject having cRORA an effective amount of an anti-C5 agent or a pharmaceutically acceptable salt thereof, wherein the progression of the ophthalmological disease, disorder, and/or condition is reduced, slowed, inhibited, prevented, improved, and/or reversed.

In one aspect, the present invention relates to methods for treating a subject having high-risk drusen, comprising administration to the subject having high-risk drusen an effective amount of an anti-C5 agent or a pharmaceutically acceptable salt thereof.

In one aspect, the present invention relates to methods of treating an ophthalmological disease, disorder, and/or condition comprising administering an effective amount of an anti-C5 agent or a pharmaceutically acceptable salt thereof to a subject in need thereof having high-risk drusen and risk factors for the progression to iRORA.

In one aspect, the present invention relates to methods of treating an ophthalmological disease, disorder, and/or condition comprising administering an effective amount of an anti-C5 agent or a pharmaceutically acceptable salt thereof to a subject in need thereof having high-risk drusen and iRORA.

In one aspect, the present invention relates to methods of treating an ophthalmological disease, disorder, and/or condition comprising administering an effective amount of an anti-C5 agent or a pharmaceutically acceptable salt thereof to a subject in need thereof having high-risk drusen and nGA.

In one aspect, the present invention relates to methods of treating an ophthalmological disease, disorder, and/or condition comprising administering an effective amount of an anti-C5 agent or a pharmaceutically acceptable salt thereof to a subject in need thereof having high-risk drusen and intermediate AMD.

In one aspect, the present invention relates to methods of treating an ophthalmological disease, disorder, and/or condition comprising administering an effective amount of an anti-C5 agent or a pharmaceutically acceptable salt thereof to a subject in need thereof having high-risk drusen and cRORA.

In one aspect, the present invention relates to methods of treating an ophthalmological disease, disorder, and/or condition comprising administering an effective amount of an anti-C5 agent or a pharmaceutically acceptable salt thereof to a subject in need thereof having high-risk drusen and GA.

In one aspect, the present invention relates to methods of treating an ophthalmological disease, disorder, and/or condition comprising administering an effective amount of an anti-C5 agent or a pharmaceutically acceptable salt thereof to a subject in need thereof having high-risk drusen and AMD.

In one aspect, the present invention relates to methods of treating an ophthalmological disease, disorder, and/or condition comprising administering an effective amount of an anti-C5 agent or a pharmaceutically acceptable salt thereof to a subject in need thereof having high-risk drusen and dry AMD.

In one aspect, the present invention relates to methods of treating an ophthalmological disease, disorder, and/or condition comprising administering an effective amount of an anti-C5 agent or a pharmaceutically acceptable salt thereof to a subject in need thereof having high-risk drusen and wet AMD.

In one aspect, the present invention relates to methods of treating an ophthalmological disease, disorder, and/or condition comprising administering an effective amount of an anti-C5 agent or a pharmaceutically acceptable salt thereof to a subject having drusen that are <63 μm in diameter.

In one aspect, the present invention relates to methods of treating an ophthalmological disease, disorder, and/or condition comprising administering an effective amount of an anti-C5 agent or a pharmaceutically acceptable salt thereof to a subject having drusen between ≥63 μm and <125 μm in diameter.

In one aspect, the present invention relates to methods of treating an ophthalmological disease, disorder, and/or condition comprising administering an effective amount of an anti-C5 agent or a pharmaceutically acceptable salt thereof to a subject having drusen of ≥125 μm in diameter.

In one aspect, the present invention relates to methods of treating an ophthalmological disease, disorder, and/or condition comprising administering an effective amount of an anti-C5 agent or a pharmaceutically acceptable salt thereof to a subject in need thereof having high-risk drusen and risk factors for the progression to iRORA, wherein the progression of the ophthalmological disease, disorder, and/or condition is reduced, slowed, inhibited, prevented, improved, and/or reversed.

In one aspect, the present invention relates to methods of treating an ophthalmological disease, disorder, and/or condition comprising administering an effective amount of an anti-C5 agent or a pharmaceutically acceptable salt thereof to a subject in need thereof having high-risk drusen and iRORA, wherein the progression of the ophthalmological disease, disorder, and/or condition is reduced, slowed, inhibited, prevented, improved, and/or reversed.

In one aspect, the present invention relates to methods of treating an ophthalmological disease, disorder, and/or condition comprising administering an effective amount of an anti-C5 agent or a pharmaceutically acceptable salt thereof to a subject in need thereof having high-risk drusen and nGA, wherein the progression of the ophthalmological disease, disorder, and/or condition is reduced, slowed, inhibited, prevented, improved, and/or reversed.

In one aspect, the present invention relates to methods of treating an ophthalmological disease, disorder, and/or condition comprising administering an effective amount of an anti-C5 agent or a pharmaceutically acceptable salt thereof to a subject in need thereof having high-risk drusen and intermediate AMD, wherein the progression of the ophthalmological disease, disorder, and/or condition is reduced, slowed, inhibited, prevented, improved, and/or reversed.

In one aspect, the present invention relates to methods of treating an ophthalmological disease, disorder, and/or condition comprising administering an effective amount of an anti-C5 agent or a pharmaceutically acceptable salt thereof to a subject having high-risk drusen and risk factors for the progression to cRORA, wherein the progression of the ophthalmological disease, disorder, and/or condition is reduced, slowed, inhibited, prevented, improved, and/or reversed.

In one aspect, the present invention relates to methods of treating an ophthalmological disease, disorder, and/or condition comprising administering an effective amount of an anti-C5 agent or a pharmaceutically acceptable salt thereof to a subject in need thereof having high-risk drusen and cRORA, wherein the progression of the ophthalmological disease, disorder, and/or condition is reduced, slowed, inhibited, prevented, improved, and/or reversed.

In one aspect, the present invention relates to methods of treating an ophthalmological disease, disorder, and/or condition comprising administering an effective amount of an anti-C5 agent or a pharmaceutically acceptable salt thereof to a subject in need thereof having high-risk drusen and GA, wherein the progression of the ophthalmological disease, disorder, and/or condition is reduced, slowed, inhibited, prevented, improved, and/or reversed.

In one aspect, the present invention relates to methods of treating an ophthalmological disease, disorder, and/or condition comprising administering an effective amount of an anti-C5 agent or a pharmaceutically acceptable salt thereof to a subject in need thereof having high-risk drusen and AMD, wherein the progression of the ophthalmological disease, disorder, and/or condition is reduced, slowed, inhibited, prevented, improved, and/or reversed.

In one aspect, the present invention relates to methods of treating an ophthalmological disease, disorder, and/or condition comprising administering an effective amount of an anti-C5 agent or a pharmaceutically acceptable salt thereof to a subject in need thereof having high-risk drusen and dry AMD, wherein the progression of the ophthalmological disease, disorder, and/or condition is reduced, slowed, inhibited, prevented, improved, and/or reversed.

In one aspect, the present invention relates to methods of treating an ophthalmological disease, disorder, and/or condition comprising administering an effective amount of an anti-C5 agent or a pharmaceutically acceptable salt thereof to a subject in need thereof having high-risk drusen and wet AMD, wherein the progression of the ophthalmological disease, disorder, and/or condition is reduced, slowed, inhibited, prevented, improved, and/or reversed.

In one aspect, the present invention relates to methods of treating an ophthalmological disease, disorder, and/or condition comprising administering an effective amount of an anti-C5 agent or a pharmaceutically acceptable salt thereof to a subject in need thereof having drusen that are <63 μm in diameter, wherein the progression of the ophthalmological disease, disorder, and/or condition is reduced, slowed, inhibited, prevented, improved, and/or reversed.

In one aspect, the present invention relates to methods of treating an ophthalmological disease, disorder, and/or condition comprising administering an effective amount of an anti-C5 agent or a pharmaceutically acceptable salt thereof to a subject in need thereof having drusen between ≥63 μm and <125 μm in diameter, wherein the progression of the ophthalmological disease, disorder, and/or condition is reduced, slowed, inhibited, prevented, improved, and/or reversed.

In one aspect, the present invention relates to methods of treating an ophthalmological disease, disorder, and/or condition comprising administering an effective amount of an anti-C5 agent or a pharmaceutically acceptable salt thereof to a subject in need thereof having drusen of ≥125 μm in diameter, wherein the progression of the ophthalmological disease, disorder, and/or condition is reduced, slowed, inhibited, prevented, improved, and/or reversed.

In one aspect, the present invention relates to methods of treating an ophthalmological disease, disorder, and/or condition comprising administering an effective amount of an anti-C5 agent or a pharmaceutically acceptable salt thereof to a subject in need thereof having high-risk drusen and risk factors for the progression to iRORA, wherein progression to iRORA is reduced, slowed, inhibited, prevented, improved, and/or reversed.

In one aspect, the present invention relates to methods of treating an ophthalmological disease, disorder, and/or condition comprising administering an effective amount of an anti-C5 agent or a pharmaceutically acceptable salt thereof to a subject in need thereof having high-risk drusen and risk factors for the progression to cRORA, wherein progression to cRORA is reduced, slowed, inhibited, prevented, improved, and/or reversed.

In one aspect, the present invention relates to methods of treating an ophthalmological disease, disorder, and/or condition comprising administering an effective amount of an anti-C5 agent or a pharmaceutically acceptable salt thereof to a subject in need thereof having high-risk drusen and risk factors for the progression to iRORA, wherein the progression to nGA is reduced, slowed, inhibited, prevented, improved, and/or reversed.

In one aspect, the present invention relates to methods of treating an ophthalmological disease, disorder, and/or condition comprising administering an effective amount of an anti-C5 agent or a pharmaceutically acceptable salt thereof to a subject in need thereof having high-risk drusen and risk factors for the progression to nGA, wherein the progression to nGA is reduced, slowed, inhibited, prevented, improved, and/or reversed.

In one aspect, the present invention relates to methods of treating an ophthalmological disease, disorder, and/or condition comprising administering an effective amount of an anti-C5 agent or a pharmaceutically acceptable salt thereof to a subject in need thereof having high-risk drusen and intermediate AMD, wherein the progression of intermediate AMD to cRORA is reduced, slowed, inhibited, prevented, improved, and/or reversed.

In one aspect, the present invention relates to methods of treating an ophthalmological disease, disorder, and/or condition comprising administering an effective amount of an anti-C5 agent or a pharmaceutically acceptable salt thereof to a subject in need thereof having high-risk drusen and iRORA, wherein the progression of iRORA to cRORA is reduced, slowed, inhibited, prevented, improved, and/or reversed.

In one aspect, the present invention relates to methods of treating an ophthalmological disease, disorder, and/or condition comprising administering an effective amount of an anti-C5 agent or a pharmaceutically acceptable salt thereof to a subject in need thereof having high-risk drusen and iRORA, wherein the progression of iRORA to GA is reduced, slowed, inhibited, prevented, improved, and/or reversed.

In one aspect, the present invention relates to methods of treating an ophthalmological disease, disorder, and/or condition comprising administering an effective amount of an anti-C5 agent or a pharmaceutically acceptable salt thereof to a subject in need thereof having high-risk drusen and nGA, wherein the progression of nGA to GA is reduced, slowed, inhibited, prevented, improved, and/or reversed.

In one aspect, the present invention relates to methods of treating an ophthalmological disease, disorder, and/or condition comprising administering an effective amount of an anti-C5 agent or a pharmaceutically acceptable salt thereof to a subject in need thereof having high-risk drusen and nGA, wherein the progression of nGA to cRORA is reduced, slowed, inhibited, prevented, improved, and/or reversed.

In one aspect, the present invention relates to methods of treating an ophthalmological disease, disorder, and/or condition comprising administering an effective amount of an anti-C5 agent or a pharmaceutically acceptable salt thereof to a subject in need thereof having high-risk drusen and cRORA, wherein the progression of cRORA to wet AMD is reduced, slowed, inhibited, prevented, improved, and/or reversed.

In one aspect, the present invention relates to methods of treating an ophthalmological disease, disorder, and/or condition comprising administering an effective amount of an anti-C5 agent or a pharmaceutically acceptable salt thereof to a subject in need thereof having high-risk drusen and GA, wherein the progression of GA to wet AMD is reduced, slowed, inhibited, prevented, improved, and/or reversed.

In one aspect, the present invention relates to methods of treating an ophthalmological disease, disorder, and/or condition comprising administering an effective amount of an anti-C5 agent or a pharmaceutically acceptable salt thereof to a subject in need thereof having high-risk drusen and dry AMD, wherein the progression of dry AMD to wet AMD is reduced, slowed, inhibited, prevented, improved, and/or reversed.

In one aspect, the present invention relates to methods of treating an ophthalmological disease, disorder, and/or condition comprising administering an effective amount of an anti-C5 agent or a pharmaceutically acceptable salt thereof to a subject in need thereof having drusen that are <63 μm in diameter, wherein the progression of AMD is reduced, slowed, inhibited, prevented, improved, and/or reversed.

In one aspect, the present invention relates to methods of treating an ophthalmological disease, disorder, and/or condition comprising administering an effective amount of an anti-C5 agent or a pharmaceutically acceptable salt thereof to a subject in need thereof having drusen between ≥63 μm and <125 μm in diameter, wherein the progression of AMD is reduced, slowed, inhibited, prevented, improved, and/or reversed.

In one aspect, the present invention relates to methods of treating an ophthalmological disease, disorder, and/or condition comprising administering an effective amount of an anti-C5 agent or a pharmaceutically acceptable salt thereof to a subject in need thereof having drusen that are ≥125 μm in diameter, wherein the progression of AMD is reduced, slowed, inhibited, prevented, improved, and/or reversed.

In one aspect, the present invention relates to methods of treating risk factors for the progression to iRORA comprising administering to a subject having risk factors for the progression to iRORA an effective amount of an anti-C5 agent or a pharmaceutically acceptable salt thereof, wherein the subject has high-risk drusen.

In one aspect, the present invention relates to methods of treating iRORA comprising administering to a subject having iRORA an effective amount of an anti-C5 agent or a pharmaceutically acceptable salt thereof, wherein the subject has high-risk drusen.

In one aspect, the present invention relates to methods of treating nGA comprising administering to a subject having nGA an effective amount of an anti-C5 agent or a pharmaceutically acceptable salt thereof, wherein the subject has high-risk drusen.

In one aspect, the present invention relates to methods of treating intermediate AMD comprising administering to a subject having intermediate AMD an effective amount of an anti-C5 agent or a pharmaceutically acceptable salt thereof, wherein the subject has high-risk drusen.

In one aspect, the present invention relates to methods of treating cRORA comprising administering to a subject having cRORA an effective amount of an anti-C5 agent or a pharmaceutically acceptable salt thereof, wherein the subject has high-risk drusen.

In one aspect, the present invention relates to methods of treating GA comprising administering to a subject having GA an effective amount of an anti-C5 agent or a pharmaceutically acceptable salt thereof, wherein the subject has high-risk drusen.

In one aspect, the present invention relates to methods of treating AMD comprising administering to a subject having AMD an effective amount of an anti-C5 agent or a pharmaceutically acceptable salt thereof, wherein the subject has high-risk drusen.

In one aspect, the present invention relates to methods of treating dry AMD comprising administering to a subject having dry AMD an effective amount of an anti-C5 agent or a pharmaceutically acceptable salt thereof, wherein the subject has high-risk drusen.

In one aspect, the present invention relates to methods of treating wet AMD comprising administering to a subject having wet AMD an effective amount of an anti-C5 agent or a pharmaceutically acceptable salt thereof, wherein the subject has high-risk drusen.

In one aspect, the present invention relates to methods of treating AMD comprising administering to a subject having drusen that are <63 μm in diameter an effective amount of an anti-C5 agent or a pharmaceutically acceptable salt thereof.

In one aspect, the present invention relates to methods of treating AMD comprising administering to a subject having drusen between ≥63 μm and <125 μm in diameter an effective amount of an anti-C5 agent or a pharmaceutically acceptable salt thereof.

In one aspect, the present invention relates to methods of treating AMD comprising administering to a subject having drusen of ≥125 μm in diameter an effective amount of an anti-C5 agent or a pharmaceutically acceptable salt thereof.

In one aspect, the present invention relates to methods of treating risk factors for the progression to iRORA comprising administering to a subject having risk factors for the progression to iRORA an effective amount of an anti-C5 agent or a pharmaceutically acceptable salt thereof, wherein the subject has high-risk drusen, wherein the progression of the ophthalmological disease, disorder, and/or condition is reduced, slowed, inhibited, prevented, improved, and/or reversed.

In one aspect, the present invention relates to methods of treating iRORA comprising administering to a subject having iRORA an effective amount of an anti-C5 agent or a pharmaceutically acceptable salt thereof, wherein the subject has high-risk drusen, wherein the progression of the ophthalmological disease, disorder, and/or condition is reduced, slowed, inhibited, prevented, improved, and/or reversed.

In one aspect, the present invention relates to methods of treating nGA comprising administering to a subject having nGA an effective amount of an anti-C5 agent or a pharmaceutically acceptable salt thereof, wherein the subject has high-risk drusen, and wherein the progression of the ophthalmological disease, disorder, and/or condition is reduced, slowed, inhibited, prevented, improved, and/or reversed.

In one aspect, the present invention relates to methods of treating intermediate AMD comprising administering to a subject having intermediate AMD an effective amount of an anti-C5 agent or a pharmaceutically acceptable salt thereof, wherein the subject has high-risk drusen, and wherein the progression of the ophthalmological disease, disorder, and/or condition is reduced, slowed, inhibited, prevented, improved, and/or reversed.

In one aspect, the present invention relates to methods of treating cRORA comprising administering to a subject having cRORA an effective amount of an anti-C5 agent or a pharmaceutically acceptable salt thereof, wherein the subject has high-risk drusen, wherein the progression of the ophthalmological disease, disorder, and/or condition is reduced, slowed, inhibited, prevented, improved, and/or reversed.

In one aspect, the present invention relates to methods of treating GA comprising administering to a subject having GA an effective amount of an anti-C5 agent or a pharmaceutically acceptable salt thereof, wherein the subject has high-risk drusen, wherein the progression of the ophthalmological disease, disorder, and/or condition is reduced, slowed, inhibited, prevented, improved, and/or reversed.

In one aspect, the present invention relates to methods of treating AMD comprising administering to a subject having AMD an effective amount of an anti-C5 agent or a pharmaceutically acceptable salt thereof, wherein the subject has high-risk drusen, wherein the progression of the ophthalmological disease, disorder, and/or condition is reduced, slowed, inhibited, prevented, improved, and/or reversed.

In one aspect, the present invention relates to methods of treating dry AMD comprising administering to a subject having dry AMD an effective amount of an anti-C5 agent or a pharmaceutically acceptable salt thereof, wherein the subject has high-risk drusen, wherein the progression of the ophthalmological disease, disorder, and/or condition is reduced, slowed, inhibited, prevented, improved, and/or reversed.

In one aspect, the present invention relates to methods of treating wet AMD comprising administering to a subject having wet AMD an effective amount of an anti-C5 agent or a pharmaceutically acceptable salt thereof, wherein the subject has high-risk drusen, wherein the progression of the ophthalmological disease, disorder, and/or condition is reduced, slowed, inhibited, prevented, improved, and/or reversed.

In one aspect, the present invention relates to methods of treating AMD comprising administering to a subject having drusen that are <63 μm in diameter an effective amount of an anti-C5 agent or a pharmaceutically acceptable salt thereof, wherein the progression of the ophthalmological disease, disorder, and/or condition is reduced, slowed, inhibited, prevented, improved, and/or reversed.

In one aspect, the present invention relates to methods of treating AMD comprising administering to a subject having drusen between ≤63 μm and <125 μm in diameter an effective amount of an anti-C5 agent or a pharmaceutically acceptable salt thereof, wherein the progression of the ophthalmological disease, disorder, and/or condition is reduced, slowed, inhibited, prevented, improved, and/or reversed.

In one aspect, the present invention relates to methods of treating AMD comprising administering to a subject having drusen of ≥125 μm in diameter an effective amount of an anti-C5 agent or a pharmaceutically acceptable salt thereof, wherein the progression of the ophthalmological disease, disorder, and/or condition is reduced, slowed, inhibited, prevented, improved, and/or reversed.

In one aspect, the present invention relates to methods of treating risk factors for the progression to iRORA comprising administering to a subject having risk factors for the progression to iRORA an effective amount of an anti-C5 agent or a pharmaceutically acceptable salt thereof, and wherein the progression to iRORA is reduced, slowed, inhibited, prevented, improved, and/or reversed.

In one aspect, the present invention relates to methods of treating risk factors for the progression to iRORA comprising administering to a subject having risk factors for the progression to iRORA an effective amount of an anti-C5 agent or a pharmaceutically acceptable salt thereof, wherein the subject has high-risk drusen, and wherein the progression to iRORA is reduced, slowed, inhibited, prevented, improved, and/or reversed.

In one aspect, the present invention relates to methods of treating risk factors for iRORA comprising administering to a subject having risk factors for iRORA an effective amount of an anti-C5 agent or a pharmaceutically acceptable salt thereof, and wherein the progression of risk factors for iRORA to iRORA is reduced, slowed, inhibited, prevented, improved, and/or reversed.

In one aspect, the present invention relates to methods of treating risk factors for iRORA comprising administering to a subject having risk factors for iRORA an effective amount of an anti-C5 agent or a pharmaceutically acceptable salt thereof, wherein the subject has high-risk drusen, and wherein the progression of risk factors for iRORA to iRORA is reduced, slowed, inhibited, prevented, improved, and/or reversed.

In one aspect, the present invention relates to methods of treating risk factors for iRORA comprising administering to a subject having risk factors for iRORA an effective amount of an anti-C5 agent or a pharmaceutically acceptable salt thereof, wherein the subject has high-risk drusen, wherein the progression of risk factors for iRORA to nGA is reduced, slowed, inhibited, prevented, improved, and/or reversed.

In one aspect, the present invention relates to methods of treating risk factors for iRORA comprising administering to a subject having iRORA an effective amount of an anti-C5 agent or a pharmaceutically acceptable salt thereof, wherein the progression of risk factors for iRORA to nGA is reduced, slowed, inhibited, prevented, improved, and/or reversed.

In one aspect, the present invention relates to methods of treating risk factors for the progression to iRORA comprising administering to a subject having risk factors for the progression to iRORA an effective amount of an anti-C5 agent or a pharmaceutically acceptable salt thereof, wherein the subject has high-risk drusen, wherein the progression of risk factors for iRORA to nGA is reduced, slowed, inhibited, prevented, improved, and/or reversed.

In one aspect, the present invention relates to methods of treating iRORA comprising administering to a subject having iRORA an effective amount of an anti-C5 agent or a pharmaceutically acceptable salt thereof, wherein the progression of iRORA to cRORA is reduced, slowed, inhibited, prevented, improved, and/or reversed.

In one aspect, the present invention relates to methods of treating iRORA comprising administering to a subject having iRORA an effective amount of an anti-C5 agent or a pharmaceutically acceptable salt thereof, wherein the subject has high-risk drusen, wherein the progression of iRORA to cRORA is reduced, slowed, inhibited, prevented, improved, and/or reversed.

In one aspect, the present invention relates to methods of treating iRORA comprising administering to a subject having iRORA an effective amount of an anti-C5 agent or a pharmaceutically acceptable salt thereof, wherein the progression of iRORA to GA is reduced, slowed, inhibited, prevented, improved, and/or reversed.

In one aspect, the present invention relates to methods of treating iRORA comprising administering to a subject having iRORA an effective amount of an anti-C5 agent or a pharmaceutically acceptable salt thereof, wherein the subject has high-risk drusen, wherein the progression of iRORA to GA is reduced, slowed, inhibited, prevented, improved, and/or reversed.

In one aspect, the present invention relates to methods of treating nGA comprising administering to a subject having nGA an effective amount of an anti-C5 agent or a pharmaceutically acceptable salt thereof, wherein the progression of nGA to GA is reduced, slowed, inhibited, prevented, improved, and/or reversed.

In one aspect, the present invention relates to methods of treating nGA comprising administering to a subject having nGA an effective amount of an anti-C5 agent or a pharmaceutically acceptable salt thereof, wherein the subject has high-risk drusen, wherein the progression of nGA to GA is reduced, slowed, inhibited, prevented, improved, and/or reversed.

In one aspect, the present invention relates to methods of treating nGA comprising administering to a subject having nGA an effective amount of an anti-C5 agent or a pharmaceutically acceptable salt thereof, wherein the progression of nGA to cRORA is reduced, slowed, inhibited, prevented, improved, and/or reversed.

In one aspect, the present invention relates to methods of treating nGA comprising administering to a subject having nGA an effective amount of an anti-C5 agent or a pharmaceutically acceptable salt thereof, wherein the subject has high-risk drusen, wherein the progression of nGA to cRORA is reduced, slowed, inhibited, prevented, improved, and/or reversed.

In one aspect, the present invention relates to methods of treating intermediate AMD comprising administering to a subject having intermediate AMD an effective amount of an anti-C5 agent or a pharmaceutically acceptable salt thereof, wherein the progression of intermediate AMD to nGA is reduced, slowed, inhibited, prevented, improved, and/or reversed.

In one aspect, the present invention relates to methods of treating intermediate AMD comprising administering to a subject having intermediate AMD an effective amount of an anti-C5 agent or a pharmaceutically acceptable salt thereof, wherein the subject has high-risk drusen, wherein the progression of intermediate AMD to nGA is reduced, slowed, inhibited, prevented, improved, and/or reversed.

In one aspect, the present invention relates to methods of treating intermediate AMD comprising administering to a subject having intermediate AMD an effective amount of an anti-C5 agent or a pharmaceutically acceptable salt thereof, wherein the progression of intermediate AMD to GA is reduced, slowed, inhibited, prevented, improved, and/or reversed.

In one aspect, the present invention relates to methods of treating intermediate AMD comprising administering to a subject having intermediate AMD an effective amount of an anti-C5 agent or a pharmaceutically acceptable salt thereof, wherein the subject has high-risk drusen, wherein the progression of intermediate AMD to GA is reduced, slowed, inhibited, prevented, improved, and/or reversed.

In one aspect, the present invention relates to methods of treating intermediate AMD comprising administering to a subject having intermediate AMD an effective amount of an anti-C5 agent or a pharmaceutically acceptable salt thereof, wherein the progression of intermediate AMD to cRORA is reduced, slowed, inhibited, prevented, improved, and/or reversed.

In one aspect, the present invention relates to methods of treating intermediate AMD comprising administering to a subject having intermediate AMD an effective amount of an anti-C5 agent or a pharmaceutically acceptable salt thereof, wherein the subject has high-risk drusen, wherein the progression of intermediate AMD to cRORA is reduced, slowed, inhibited, prevented, improved, and/or reversed.

In one aspect, the present invention relates to methods of treating cRORA comprising administering to a subject having cRORA an effective amount of an anti-C5 agent or a pharmaceutically acceptable salt thereof, wherein the progression of cRORA to wet AMD is reduced, slowed, inhibited, prevented, improved, and/or reversed.

In one aspect, the present invention relates to methods of treating cRORA comprising administering to a subject having cRORA an effective amount of an anti-C5 agent or a pharmaceutically acceptable salt thereof, wherein the subject has high-risk drusen, wherein the progression of cRORA to wet AMD is reduced, slowed, inhibited, prevented, improved, and/or reversed.

In one aspect, the present invention relates to methods of treating intermediate AMD comprising administering to a subject having drusen with a diameter greater than 125 μm and/or iRORA and/or hyperreflective foci an effective amount of an anti-C5 agent or a pharmaceutically acceptable salt thereof, wherein the progression of intermediate AMD to cRORA is reduced, slowed, inhibited, prevented, improved, and/or reversed.

In one aspect, the present invention relates to methods of treating intermediate AMD comprising administering to a subject having drusen with a diameter greater than 125 μm and/or iRORA and/or hyperreflective foci an effective amount of an anti-C5 agent or a pharmaceutically acceptable salt thereof, wherein the subject has high-risk drusen, wherein the progression of intermediate AMD to cRORA is reduced, slowed, inhibited, prevented, improved, and/or reversed.

In one aspect, the present invention relates to methods of treating intermediate AMD comprising administering to a subject having intermediate AMD an effective amount of an anti-C5 agent or a pharmaceutically acceptable salt thereof, wherein the progression of intermediate AMD to wet AMD is reduced, slowed, inhibited, prevented, improved, and/or reversed.

In one aspect, the present invention relates to methods of treating intermediate AMD comprising administering to a subject having intermediate AMD an effective amount of an anti-C5 agent or a pharmaceutically acceptable salt thereof, wherein the subject has high-risk drusen, wherein the progression of intermediate AMD to wet AMD is reduced, slowed, inhibited, prevented, improved, and/or reversed.

In one aspect, the present invention relates to methods of treating GA comprising administering to a subject having GA an effective amount of an anti-C5 agent or a pharmaceutically acceptable salt thereof, wherein the subject has high-risk drusen, wherein the progression of GA to wet AMD is reduced, slowed, inhibited, prevented, improved, and/or reversed.

In one aspect, the present invention relates to methods of treating dry AMD comprising administering to a subject having dry AMD an effective amount of an anti-C5 agent or a pharmaceutically acceptable salt thereof, wherein the subject has high-risk drusen, wherein the progression of dry AMD to wet AMD is reduced, slowed, inhibited, prevented, improved, and/or reversed.

In one aspect, the present invention relates to methods of treating early-stage AMD comprising administering to a subject having drusen that are <63 μm in diameter an effective amount of an anti-C5 agent or a pharmaceutically acceptable salt thereof, wherein the subject has high-risk drusen, wherein the progression of drusen that are <63 μm in diameter to drusen that are drusen between ≥63 μm and <125 μm in diameter is reduced, slowed, inhibited, prevented, improved, and/or reversed.

In one aspect, the present invention relates to methods of treating early-stage AMD comprising administering to a subject having drusen that are between ≥63 μm and <125 μm in diameter an effective amount of an anti-C5 agent or a pharmaceutically acceptable salt thereof, wherein the subject has high-risk drusen, wherein the progression of drusen that are drusen between ≥63 μm and <125 μm in diameter to drusen of ≥125 μm in diameter is reduced, slowed, inhibited, prevented, improved, and/or reversed.

In another aspect, administration of an anti-C5 agent or a pharmaceutically acceptable salt thereof according to the methods of the invention reduce, slow, inhibit, prevent, improve, and/or reverse at least one characteristic of drusen (e.g., number, extent, area, volume, shape, density, and reflectivity), such as in subjects having an ophthalmological disease, disorder, and/or condition selected from the group consisting of risk factors for the progression to iRORA, iRORA, nGA, cRORA, GA, AMD, intermediate AMD, dry AMD, and/or wet AMD. In some embodiments, the reduction, slowing, inhibition, prevention, improvement, and/or reversal of at least one characteristic of drusen (e.g., number, extent, area, volume, shape, density, and reflectivity) is accompanied by the lack of obvious sequelae, AMD disease progression, and/or conversion to a more advanced disease state.

In some embodiments, the administration of an anti-C5 agent or a pharmaceutically acceptable salt thereof may reduce, inhibit, prevent, slow, and/or reverse one or more symptoms of nGA secondary to AMD, including, but not limited to, reduced or loss of central vision, loss of visual acuity, and reduced or inability to read. In some embodiments, the administration of an anti-C5 agent or a pharmaceutically acceptable salt thereof may inhibit, prevent, slow, and/or reverse one or more of tissue- or cellular-level changes that is associated with nGA secondary to AMD, including, but not limited to, growth of nGA lesions, rate of growth or change in nGA lesions, and the formation of drusen under the RPE.

In some embodiments, the administration of an anti-C5 agent or a pharmaceutically acceptable salt thereof may reduce, inhibit, prevent, slow, and/or reverse one or more symptoms of GA secondary to AMD, including, but not limited to, reduced or loss of central vision, loss of visual acuity, and reduced or inability to read. In some embodiments, the administration of an anti-C5 agent or a pharmaceutically acceptable salt thereof may inhibit, prevent, slow, and/or reverse one or more of tissue- or cellular-level changes that is associated with GA secondary to AMD, including, but not limited to, growth of GA lesions, rate of growth or change in GA lesions, and the formation of drusen under the RPE.

In some embodiments, the administration of an anti-C5 agent or a pharmaceutically acceptable salt thereof is administered intravitreally.

In some embodiments, methods of treating an ophthalmological disease, disorder, and/or condition comprise administering an effective amount of an anti-C5 agent or a pharmaceutically acceptable salt thereof to a subject in need thereof having: (i) high-risk drusen and (ii) an ophthalmological disease, disorder, and/or condition selected from the group consisting of risk factors for the progression to iRORA, iRORA, nGA, cRORA, GA, AMD, intermediate AMD, dry AMD, and/or wet AMD, wherein the progression of the ophthalmological disease, disorder, and/or condition is reduced, slowed, inhibited, prevented, improved, and/or reversed, and/or wherein at least one characteristic of drusen (e.g., number, extent, area, volume, shape, density, and reflectivity) is reduced, slowed, inhibited, prevented, improved and/or reversed. In certain embodiments, the reduction, slowing, inhibition, prevention, improvement, and/or reversal includes the reduction in the rate of conversion from one disease state to a more advanced disease state. In some embodiments, the reduction, slowing, inhibition, prevention, improvement, and/or reversal includes the reduction in the rate of conversion from early- to late-stage AMD. In some embodiments, reduction, slowing, inhibition, prevention, improvement, and/or reversal may be determined by comparing morphological features of an eye over a period of time, as compared to those features in a subject who is not administered an anti-C5 agent or a pharmaceutically acceptable salt thereof, or compared to a baseline or a previous state in the subject themselves. In such embodiments, such a comparison may be made at a baseline, initial, or earlier administration of the anti-C5 agent or a pharmaceutically acceptable salt thereof and at least or about one month, at least or about two months, at least or about three months, at least or about four months, at least or about five months, at least or about six months, at least or about seven months, at least or about eight months, at least or about nine months, at least or about 10 months, at least or about 11 months, at least or about 12 months, at least or about 15 months, at least or about 18 months, or at least or about 24 months.

In some embodiments, methods of treating an ophthalmological disease, disorder, and/or condition selected from the group consisting of risk factors for the progression to iRORA, iRORA, nGA, cRORA, GA, AMD, intermediate AMD, dry AMD, and/or wet AMD, comprise administering to a subject in need thereof an effective amount of an anti-C5 agent or a pharmaceutically acceptable salt thereof, wherein the progression of the ophthalmological disease, disorder, and/or condition is reduced, slowed, inhibited, prevented, improved, and/or reversed, and/or wherein at least one characteristic of drusen (e.g., number, extent, area, volume, shape, density, and reflectivity) is reduced, slowed, inhibited, prevented, improved and/or reversed. In certain embodiments, the reduction, slowing, inhibition, prevention, improvement, and/or reversal includes the reduction in the rate of conversion from one disease state to a more advanced disease state. In some embodiments, the reduction, slowing, inhibition, prevention, improvement, and/or reversal includes the reduction in the rate of conversion from early- to late-stage AMD. In some embodiments, reduction, slowing, inhibition, prevention, improvement, and/or reversal may be determined by comparing morphological features of an eye over a period of time, as compared to those features in a subject who is not administered an anti-C5 agent or a pharmaceutically acceptable salt thereof, or compared to a baseline or a previous state in the subject themselves. In such embodiments, such a comparison may be made at a baseline, initial, or earlier administration of the anti-C5 agent or a pharmaceutically acceptable salt thereof and at least or about one month, at least or about two months, at least or about three months, at least or about four months, at least or about five months, at least or about six months, at least or about seven months, at least or about eight months, at least or about nine months, at least or about 10 months, at least or about 11 months, at least or about 12 months, at least or about 15 months, at least or about 18 months, or at least or about 24 months.

In some embodiments, an anti-C5 agent or a pharmaceutically acceptable salt thereof is administered in an amount effective to reduce the rate of drusen growth in subjects having high-risk drusen as compared to the rate of drusen growth prior to administration of an anti-C5 agent or a pharmaceutically acceptable salt thereof, or compared to a baseline or a previous state in the subject themselves. In certain embodiments, the rate of drusen growth is reduced by at least or about 5%, at least or about 10%, at least or about 15%, at least or about 20%, at least or about 25%, at least or about 30%, at least or about 35%, at least or about 40%, at least or about 45%, or at least or about 50%. Such a reduction of the rate of drusen growth may be determined by measuring how much drusen is formed under the retina over a period of time, such as between baseline and at least or about one month, at least or about two months, at least or about three months, at least or about four months, at least or about five months, at least or about six months, at least or about seven months, at least or about eight months, at least or about nine months, at least or about 10 months, at least or about 11 months, at least or about 12 months, at least or about 15 months, at least or about 18 months, or at least or about 24 months. In another embodiment, an anti-C5 agent or a pharmaceutically acceptable salt thereof is administered in an amount effective to reduce the rate of drusen growth in subjects having drusen that are <63 μm in diameter, between ≥63 μm and <125 μm in diameter, or ≥125 μm in diameter.

In some embodiments, an anti-C5 agent or a pharmaceutically acceptable salt thereof is administered in an amount effective to maintain about the same level of retinal drusen or reduce the level of retinal drusen (e.g., amount, size, number, area volume, shape, density, and/or reflectivity) in subjects having high-risk drusen as compared to the level of retinal drusen prior to administration of an anti-C5 agent or a pharmaceutically acceptable salt thereof, or compared to a baseline or a previous state in the subject themselves. In certain embodiments, the level of drusen is reduced by at least or about 5%, at least or about 10%, at least or about 15%, at least or about 20%, at least or about 25%, at least or about 30%, at least or about 35%, at least or about 40%, at least or about 45%, or at least or about 50%. Such a reduction of the level of drusen may be determined by measuring how much drusen is formed under the retina over a period of time, such as between baseline and at least or about one month, at least or about two months, at least or about three months, at least or about four months, at least or about five months, at least or about six months, at least or about seven months, at least or about eight months, at least or about nine months, at least or about 10 months, at least or about 11 months, at least or about 12 months, at least or about 15 months, at least or about 18 months, or at least or about 24 months. In another embodiment, an anti-C5 agent or a pharmaceutically acceptable salt thereof is administered in an amount effective to maintain about the same level of retinal drusen or reduce the level of retinal drusen (e.g., amount, size, number, area volume, shape, density, and/or reflectivity) in subjects having drusen that are <63 μm in diameter, between ≥63 μm and <125 μm in diameter, or ≥125 μm in diameter.

In one embodiment, an anti-C5 agent or a pharmaceutically acceptable salt thereof is administered in an amount effective to slow and/or inhibit the progression of drusen (e.g., amount, size, number, area, volume, shape, density, reflectivity, and/or morphology) in subjects having high-risk drusen as compared to the level of retinal drusen prior to administration of an anti-C5 agent or a pharmaceutically acceptable salt thereof, or compared to a baseline or a previous state in the subject themselves. In a particular embodiment, the progression of drusen is reduced by at least or about 5%, at least or about 10%, at least or about 15%, at least or about 20%, at least or about 25%, at least or about 30%, at least or about 35%, at least or about 40%, at least or about 45%, or at least or about 50%. The slowing and/or inhibiting of the progression may be determined by measuring how much drusen is formed under the retina over a period of time, such as between baseline and at least or about one month, at least or about two months, at least or about three months, at least or about four months, at least or about five months, at least or about six months, at least or about seven months, at least or about eight months, at least or about nine months, at least or about 10 months, at least or about 11 months, at least or about 12 months, at least or about 15 months, at least or about 18 months, or at least or about 24 months. In another embodiment, an anti-C5 agent or a pharmaceutically acceptable salt thereof is administered in an amount effective to slow and/or inhibit the progression of drusen (e.g., amount, size, number, area, volume, shape, density, reflectivity, and/or morphology) in subjects having drusen that are <63 μm in diameter, between ≥63 μm and <125 μm in diameter, or ≥125 μm in diameter.

In one embodiment, an anti-C5 agent as described herein is administered in an amount effective to reduce the formation of drusen, and/or reduce the rate of the formation of drusen, and/or reverse the formation of drusen in subjects having high-risk drusen as compared to the level of retinal drusen prior to administration of an anti-C5 agent or a pharmaceutically acceptable salt thereof, or compared to a baseline or a previous state in the subject themselves. In a particular embodiment, the formation of drusen and/or the rate of formation of drusen is reduced by at least or about 5%, at least or about 10%, at least or about 15%, at least or about 20%, at least or about 25%, at least or about 30%, at least or about 35%, at least or about 40%, at least or about 45%, or at least or about 50%. The reduction of the formation of drusen and/or the reduction of the rate of the formation of drusen may be determined by measuring how much drusen is formed under the retina over a period of time, such as between baseline and at least or about one month, at least or about two months, at least or about three months, at least or about four months, at least or about five months, at least or about six months, at least or about seven months, at least or about eight months, at least or about nine months, at least or about 10 months, at least or about 11 months, at least or about 12 months, at least or about 15 months, at least or about 18 months, or at least or about 24 months. In a particular embodiment, the reversal of the formation of drusen is reversed by at least or about 5%, at least or about 10%, at least or about 15%, at least or about 20%, at least or about 25%, at least or about 30%, at least or about 35%, at least or about 40%, at least or about 45%, or at least or about 50%, as compared to that in a subject who is not administered an anti-C5 agent or a pharmaceutically acceptable salt thereof. A reversal in the formation of drusen may be determined by measuring how much drusen is present under the retina after a period of time, such as after a period of time between baseline and at least or about one month, at least or about two months, at least or about three months, at least or about four months, at least or about five months, at least or about six months, at least or about seven months, at least or about eight months, at least or about nine months, at least or about 10 months, at least or about 11 months, at least or about 12 months, at least or about 15 months, at least or about 18 months, or at least or about 24 months. In another embodiment, an anti-C5 agent as described herein is administered in an amount effective to reduce the formation of drusen, and/or reduce the rate of the formation of drusen, and/or reverse the formation of drusen in subjects having drusen that are <63 μm in diameter, between ≥63 μm and <125 μm in diameter, or ≥125 μm in diameter.

In one aspect, the present invention relates to methods of slowing and/or inhibiting loss of visual acuity in a subject with high-risk drusen, comprising administering to the subject an anti-C5 agent as described herein. In some embodiments, administration of an anti-C5 agent or a pharmaceutically acceptable salt thereof to the subject may slow and/or inhibit the decrease in best corrected visual acuity (measured using Early Treatment of Diabetic Retinopathy Study (ETDRS) letters), as compared to that in a subject who is not administered an anti-C5 agent or a pharmaceutically acceptable salt thereof, or compared to a baseline or a previous state in the subject themselves. Such a decrease may be measured between baseline and at least or about one month, at least or about two months, at least or about three months, at least or about four months, at least or about five months, at least or about six months, at least or about seven months, at least or about eight months, at least or about nine months, at least or about 10 months, at least or about 11 months, at least or about 12 months, at least or about 15 months, at least or about 18 months, or at least or about 24 months. In another aspect, the present invention relates to methods of slowing and/or inhibiting loss of visual acuity in a subject having drusen that are <63 μm in diameter, between ≥63 μm and <125 μm in diameter, or ≥125 μm in diameter.

In one aspect, the present invention relates to methods of reversing the loss of visual acuity in a subject with high-risk drusen, comprising administering to the subject an anti-C5 agent as described herein. In some embodiments, administration of an anti-C5 agent or a pharmaceutically acceptable salt thereof to the subject may increase the best corrected visual acuity (measured using ETDRS), as compared to that in a subject who is not administered an anti-C5 agent or a pharmaceutically acceptable salt thereof, or compared to a baseline or a previous state in the subject themselves. Such an increase may be measured between baseline and at least or about one month, at least or about two months, at least or about three months, at least or about four months, at least or about five months, at least or about six months, at least or about seven months, at least or about eight months, at least or about nine months, at least or about 10 months, at least or about 11 months, at least or about 12 months, at least or about 15 months, at least or about 18 months, or at least or about 24 months. In another aspect, the present invention relates to methods of reversing the loss of visual acuity in a subject having drusen that are <63 μm in diameter, between ≥63 μm and ≥125 μm in diameter, or ≥125 μm in diameter.

In one aspect, the present invention relates to methods of slowing and/or inhibiting loss of low luminance visual acuity in a subject with high-risk drusen, comprising administering to the subject an anti-C5 agent as described herein. In some embodiments, administering the anti-C5 agent to the subject may slow or inhibit the decrease in low luminance best corrected visual acuity (measured using ETDRS letters), as compared to that in a subject who is not administered the anti-C5 agent, or compared to a baseline or a previous state in the subject themselves. Such a decrease may be measured between baseline and at least or about one month, at least or about two months, at least or about three months, at least or about four months, at least or about five months, at least or about six months, at least or about seven months, at least or about eight months, at least or about nine months, at least or about 10 months, at least or about 11 months, at least or about 12 months, at least or about 15 months, at least or about 18 months, or at least or about 24 months. In another aspect, the present invention relates to methods of slowing and/or inhibiting loss of low luminance visual acuity in a subject with drusen that are <63 μm in diameter, between ≥63 μm and <125 μm in diameter, or ≥125 μm in diameter.

In one aspect, the present invention relates to methods of reversing the loss of low luminance visual acuity in a subject having high-risk drusen, comprising administering to the subject an anti-C5 agent. In some embodiments, administration of the anti-C5 agent to the subject may increase the low luminance best corrected visual acuity (measured using ETDRS), as compared to that in a subject who is not administered the anti-C5 agent, or compared to a baseline or a previous state in the subject themselves. Such an increase may be measured between baseline and at least or about one month, at least or about two months, at least or about three months, at least or about four months, at least or about five months, at least or about six months, at least or about seven months, at least or about eight months, at least or about nine months, at least or about 10 months, at least or about 11 months, at least or about 12 months, at least or about 15 months, at least or about 18 months, or at least or about 24 months. In another aspect, the present invention relates to methods of reversing the loss of low luminance visual acuity in a subject having drusen that are <63 μm in diameter, between ≥63 μm and <125 μm in diameter, or ≥125 μm in diameter.

In some embodiments, the measurement using ETDRS letters may be as described in Early Treatment Diabetic Retinopathy Study Research Group (ETDRS), Manual of Operations, Baltimore. ETDRS Coordinating Center, University of Maryland. Available from: National Technical Information Service, 5285 Port Royal Road, Springfield, VA 22161; Accession No. PB8S 223006/AS; Ferris et al., Am J Ophthalmol 94:91-96, 1982. In some embodiments, the vision testing uses one or more charts available from https://www.ophthalmologyweb.com/Neuro-Ophthalmology/5650-ETDRS-Visual-Acuity-Charts/Compare/?compare=55134,49475,55138,55135,55420&catid=5650, e.g., ETDRS visual acuity Chart 1, 2 and/or R.

In some embodiments, the methods described herein may further comprise identifying the subject to be treated, such as by determining whether the subject has high-risk drusen, or has risk factors for the progression to iRORA, or has iRORA, or has nGA, or has cRORA, or has AMD, or has intermediate AMD, or has dry AMD, or has wet AMD, or has GA secondary to AMD.

In some embodiments, the subject may be a mammal, which includes, but is not limited to, a human, monkey, cow, hog, sheep, horse, dog, cat, rabbit, rat, and mouse. In certain embodiments, the subject is a human.

In some embodiments, the subject may be treatment-naïve, e.g., was not previously treated for the high-risk drusen, or risk factors for the progression to iRORA, or iRORA, or nGA, or cRORA, or AMD, or intermediate AMD, or dry AMD, or wet AMD, or GA secondary to AMD.

In some embodiments, therapeutic effectiveness may take into account a mosaic of one or more clinical endpoints, such as progression of disease state, drusen characteristics, and/or pigmentation changes. In some aspects, the therapeutic effectiveness will be determined using a scale, score, and/or statistical analyses.

### Compositions for Therapeutic or Prophylactic Administration

The anti-C5 agent can be administered as a component of a composition that further comprises a pharmaceutically acceptable carrier or vehicle, e.g., a pharmaceutical composition. The anti-C5 agent, for example, can be admixed with a suitable carrier substance, and is generally present in an amount of 1-95% by weight of the total weight of the composition. The composition may be provided in a dosage form that is suitable for injection, in particular suitable for injection directly in the eye (e.g., intravitreal injection). The composition may be in form of, for example, suspensions, emulsions, or solutions.

Formulations for injection include sterile aqueous or non-aqueous solutions, suspensions, or emulsions. A variety of aqueous carriers can be used, e.g., water, buffered water, saline, and the like. Such formulations may also contain excipients such as preserving agents, wetting agents, buffering agents, emulsifying agents, dispersing agents, and suspending agents.

In some embodiments, excipients for compositions that comprise the anti-C5 agent include, but are not limited to, buffering agents, nonionic surfactants, preservatives, tonicity agents, sugars, amino acids, and pH-adjusting agents. Suitable buffering agents include, but are not limited to, monobasic sodium phosphate, dibasic sodium phosphate, and sodium acetate. Suitable nonionic surfactants include, but are not limited to, polyoxyethylene sorbitan fatty acid esters such as polysorbate 20 and polysorbate 80. Suitable preservatives include, but are not limited to, benzyl alcohol. Suitable tonicity agents include, but are not limited to sodium chloride, mannitol, and sorbitol. Suitable sugars include, but are not limited to, α,α-trehalose. Suitable amino acids include, but are not limited, to glycine and histidine. Suitable pH-adjusting agents include, but are not limited to, hydrochloric acid, acetic acid, and sodium hydroxide. In some embodiments, the pH-adjusting agent or agents are present in an amount effective to provide a pH of about 3 to about 8, about 6 to about 8, about 6.5 to about 8, about 4 to about 7, about 5 to about 6, about 6 to about 7, or about 7 to about 7.5. In certain embodiments, the pH-adjusting agent or agents are present in an amount effective to provide a pH of about 6.8 to about 7.8. In some embodiments, the compositions do not comprise a preservative. In some embodiments, the composition does not comprise an antimicrobial agent. In some embodiments, the composition does not comprise a bacteriostat.

In certain embodiments, the composition comprises sodium chloride, sodium phosphate monobasic (monohydrate), and sodium phosphate dibasic (heptahydrate), in which the pH of the composition is about 6.8 to about 7.8.

In some embodiments, the concentration of the anti-C5 agent in a composition is about 0.5 mg/mL to about 100 mg/mL. In some embodiments, the concentration of the anti-C5 agent in a composition is less than or about 100 mg/mL, less than about 50 mg/mL, less than about 40 mg/mL, less than about 30 mg/mL, less than about 25 mg/mL, less than about 20 mg/mL, less than about 15 mg/mL, less than about 10 mg/mL, or less than about 5 mg/mL. In certain embodiments, the concentration of the anti-C5 agent in a composition is about 0.3 mg/mL to about 100 mg/mL, about 0.3 mg/mL to about 50 mg/mL, about 1 mg/mL to about 50 mg/mL, about 5 mg/mL to about 40 mg/mL, about 10 to about 30 mg/mL, about 15 mg/mL to about 25 mg/mL, about 1 mg/mL, about 5 mg/mL, about 10 mg/mL, about 15 mg/mL, about 20 mg/mL, about 25 mg/mL, about 30 mg/mL, about 35 mg/mL, about 40 mg/mL, about 45 mg/mL, or about 50 mg/mL. In particular embodiments, the concentration of the anti-C5 agent in a composition is about 20 mg/mL.

### Administration and Dosage

The dosage of the anti-C5 agent for administration to the eye may be about 0.5 mg/eye to about 5 mg/eye, or about 1 mg/eye to about 4 mg/eye, or about 2 mg/eye to about 4 mg/eye, about 1 mg/eye, or about 2 mg/eye, or about 3 mg/eye, or about 4 mg/eye. The anti-C5 agent may be administered ocularly, for example by intravitreal injection. The anti-C5 agent may be administered once a week, once every two weeks, once every three weeks, once every four weeks, once every five weeks, once every six weeks, once every seven weeks, once every eight weeks, once every nine weeks, once every 10 weeks, once every 11 weeks, or once every 12 weeks. The dosages may be administered once a month, once every two months (e.g., once every other month), once every three months, once every four months, once every five months, once every six months, once every seven months, once every eight months, once every nine months, once every ten months, once every eleven months, or once every twelve months. In certain embodiments, the dosages are administered once a month. In certain embodiments, the dosages are administered once a year.

In embodiments of the invention, the anti-C5 agent may be administered in a dosing regimen comprising a loading phase and maintenance phase. In some embodiments, the loading phase may comprise administering the anti-C5 agent at a different dosage, at a different frequency, or a combination thereof, as compared to the maintenance phase. For instance, the loading phase may comprise administering the anti-C5 agent at a dose of about 0.5 mg/eye, or about 1 mg/eye, or about 2 mg/eye, or about 3 mg/eye, or about 4 mg/eye; and the maintenance phase may comprise administering the anti-C5 agent at a dose that is a percentage of, or greater than, the dose of the anti-C5 agent of the loading phase, such as about 10%, or about 20%, or about 25%, or about 30%, or about 33%, or about 40%, or about 50%, or about 60%, or about 67%, or about 70%, or about 75%, or about 80%, or about 90%, or about 100%, or about 125%, or about 150%, or about 175%, or about 200%, or about 225%, or about 250%, or about 275%, or about 300%, or about 325%, or about 350%, or about 375%, or about 400%, of the dose of the anti-C5 agent of the loading phase. Alternatively, or in addition, the loading phase may comprise administering the anti-C5 agent at a frequency in which the duration between doses is one week, two weeks, three weeks, four weeks, one month, five weeks, six weeks, seven weeks, eight weeks, two months, nine weeks, 10 weeks, 11 weeks, 12 weeks, three months, four months, five months, or six months; and the maintenance phase may comprise administering the anti-C5 agent at a frequency in which the duration between doses is a percentage of, or greater than, the duration between doses of the loading phase, such as about 10%, or about 20%, or about 25%, or about 30%, or about 33%, or about 40%, or about 50%, or about 60%, or about 67%, or about 70%, or about 75%, or about 80%, or about 90%, or about 100%, or about 125%, or about 150%, or about 175%, or about 200%, or about 225%, or about 250%, or about 275%, or about 300%, or about 325%, or about 350%, or about 375%, or about 400%, of the duration between doses of the loading phase. In some embodiments, the loading phase may last a duration of about one week, about two weeks, about three weeks, about four weeks, about one month, about five weeks, about six weeks, about seven weeks, about eight weeks, about two months, about nine weeks, about 10 weeks, about 11 weeks, about 12 weeks, about three months, about four months, about six months, about eight months, about nine months, about 12 months, about 15 months, about 18 months, about 21 months, or about 24 months; and the maintenance phase may begin at the conclusion of the loading phase.

In certain embodiments, the anti-C5 agent may be administered in a dosing regimen comprising a loading phase that comprises a dose of about 2 mg/eye administered once a month for a duration of one year, followed by a maintenance phase that comprises a dose of about 2 mg/eye administered once every two months. In certain embodiments, the anti-C5 agent may be administered in a dosing regimen comprising a loading phase that comprises a dose of about 4 mg/eye administered once a month for a duration of one year, followed by a maintenance phase that comprises a dose of about 2 mg/eye administered once a month. In certain embodiments, the anti-C5 agent may be administered in a dosing regimen comprising a loading phase that comprises a dose of about 2 mg/eye administered once a month for a duration of about six months, followed by a maintenance phase that comprises a dose of about 2 mg/eye administered once every two months.

The amount of the composition comprising the anti-C5 agent administered to the subject can range from about 0.01 mL to about 0.2 mL administered per eye, or about 0.03 mL to about 0.15 mL administered per eye, or about 0.05 mL to about 0.10 mL administered per eye.

In some embodiments, the subject may receive more than one injection per eye of a lower dosage. For example, the subject may receive two injections of a 2-mg dose, rather than a single 4-mg dose.

### Other Aspects of the Present Invention

In some embodiments, the present invention relates to the anti-C5 agent as described herein for use in treating subjects having risk factors for progression to iRORA; subjects having iRORA; subjects having nGA; subjects having intermediate AMD; subjects having cRORA; subjects having high-risk drusen; slowing, inhibiting, preventing, and/or reversing the progression of high-risk drusen to dry AMD; slowing, inhibiting, preventing, and/or reversing the progression of nGA to GA secondary to AMD; slowing, inhibiting, preventing, and/or reversing the progression of intermediate AMD to cRORA; slowing, inhibiting, preventing, and/or reversing the progression of dry AMD to wet AMD; slowing, inhibiting, preventing, and/or reversing the progression of iRORA to wet AMD; slowing, inhibiting, preventing, and/or reversing the progression of cRORA to wet AMD; and slowing, inhibiting, preventing, and/or reversing the progression of nGA to GA secondary to AMD; slowing, inhibiting, preventing, and/or reversing the progression of high-risk drusen in a subject in need thereof; slowing, inhibiting, preventing, and/or reversing the progression of intermediate AMD in a subject in need thereof; slowing, inhibiting, preventing, and/or reversing the progression of dry AMD in a subject in need thereof; slowing, inhibiting, preventing, and/or reversing the progression of GA in a subject in need thereof; slowing, inhibiting, preventing, and/or reversing loss of visual acuity in a subject with high-risk drusen; slowing, inhibiting, preventing, and/or reversing loss of visual acuity in a subject in need thereof; slowing, inhibiting, preventing, and/or reversing loss of low luminance visual acuity in a subject with high-risk drusen: slowing, inhibiting, preventing, and/or reversing loss of low luminance visual acuity in a subject in need thereof; treating high risk drusen in a subject; reducing the rate of drusen growth in a subject; reducing the amount of drusen in a subject; reducing the area of drusen in a subject; slowing, inhibiting, preventing, and/or reversing the progression of drusen in a subject; reducing the formation of drusen in a subject; reducing the rate of formation of drusen in a subject; reversing the formation of drusen in a subject. Such uses are performed in accordance with the methods of the present invention described herein.

In some embodiments, the present invention relates to uses of the anti-C5 agent as described herein to treat subjects having risk factors for progression to iRORA; subjects having iRORA; subjects having nGA; subjects having intermediate AMD; subjects having cRORA: subjects having high-risk drusen; slow, inhibit, prevent, and/or reverse the progression of high-risk drusen to dry AMD; slow, inhibit, prevent, and/or reverse the progression of nGA to GA secondary to AMD; slow, inhibit, prevent, and/or reverse the progression of intermediate AMD to cRORA; slow, inhibit, prevent, and/or reverse the progression of dry AMD to wet AMD; slow, inhibit, prevent, and/or reverse the progression of iRORA to wet AMD; slow, inhibit, prevent, and/or reverse the progression of cRORA to wet AMD; and slow, inhibit, prevent, and/or reverse the progression of nGA to GA secondary to AMD; slow, inhibit, prevent, and/or reverse the progression of high-risk drusen in a subject in need thereof; slow, inhibit, prevent, and/or reverse the progression of intermediate AMD in a subject in need thereof; slow, inhibit, prevent, and/or reverse the progression of dry AMD in a subject in need thereof; slow, inhibit, prevent, and/or reverse the progression of GA in a subject in need thereof; slow, inhibit, prevent, and/or reverse loss of visual acuity in a subject with high-risk drusen; slow, inhibit, prevent, and/or reverse loss of visual acuity in a subject in need thereof; slow, inhibit, prevent, and/or reverse loss of low luminance visual acuity in a subject with high-risk drusen; slow, inhibit, prevent, and/or reverse loss of low luminance visual acuity in a subject in need thereof; treat high risk drusen in a subject; reduce the rate of drusen growth in a subject; reduce the amount of drusen in a subject; reduce the area of drusen in a subject; slow, inhibit, prevent, and/or reverse the progression of drusen in a subject: reduce the formation of drusen in a subject; reduce the rate of formation of drusen in a subject; reverse the formation of drusen in a subject. Such uses are performed in accordance with the methods of the present invention described herein.

In some embodiments, the present invention relates to the use of the anti-C5 agent in the manufacture of a medicament for treating subjects having risk factors for progression to iRORA; subjects having iRORA; subjects having nGA; subjects having intermediate AMD; subjects having cRORA; subjects having high-risk drusen; slowing, inhibiting, preventing, and/or reversing the progression of high-risk drusen to dry AMD; slowing, inhibiting, preventing, and/or reversing the progression of nGA to GA secondary to AMD; slowing, inhibiting, preventing, and/or reversing the progression of intermediate AMD to cRORA; slowing, inhibiting, preventing, and/or reversing the progression of dry AMD to wet AMD; slowing, inhibiting, preventing, and/or reversing the progression of iRORA to wet AMD; slowing, inhibiting, preventing, and/or reversing the progression of cRORA to wet AMD; and slowing, inhibiting, preventing, and/or reversing the progression of nGA to GA secondary to AMD; slowing, inhibiting, preventing, and/or reversing the progression of high-risk drusen in a subject in need thereof; slowing, inhibiting, preventing, and/or reversing the progression of intermediate AMD in a subject in need thereof; slowing, inhibiting, preventing, and/or reversing the progression of dry AMD in a subject in need thereof; slowing, inhibiting, preventing, and/or reversing the progression of GA in a subject in need thereof; slowing, inhibiting, preventing, and/or reversing loss of visual acuity in a subject with high-risk drusen; slowing, inhibiting, preventing, and/or reversing loss of visual acuity in a subject in need thereof; slowing, inhibiting, preventing, and/or reversing loss of low luminance visual acuity in a subject with high-risk drusen; slowing, inhibiting, preventing, and/or reversing loss of low luminance visual acuity in a subject in need thereof; treating high risk drusen in a subject; reducing the rate of drusen growth in a subject; reducing the amount of drusen in a subject; reducing the area of drusen in a subject; slowing, inhibiting, preventing, and/or reversing the progression of drusen in a subject; reducing the formation of drusen in a subject; reducing the rate of formation of drusen in a subject; reversing the formation of drusen in a subject. These uses of the medicament are performed in accordance with the methods of the present invention described herein.

In some embodiments, the present invention relates to the use of the anti-C5 agent in the manufacture of a medicament for treating a subject in need thereof having: (i) high-risk drusen and (ii) an ophthalmological disease, disorder, and/or condition selected from the group consisting of risk factors for the progression to iRORA, iRORA, nGA, cRORA, GA, AMD, intermediate AMD, dry AMD, and/or wet AMD, wherein the progression of the ophthalmological disease, disorder, and/or condition is reduced, slowed, inhibited, prevented, improved, and/or reversed. In some of the above embodiments, at least one characteristic of drusen (e.g., number, extent, area, volume, shape, density, and reflectivity) is reduced, slowed, inhibited, prevented, improved and/or reversed.

In some embodiments, the present invention relates to the use of the anti-C5 agent in the manufacture of a medicament for treating risk factors for the progression to iRORA, iRORA, nGA, cRORA, GA, AMD, intermediate AMD, dry AMD, and/or wet AMD, comprising administering to a subject having in need thereof an effective amount of an anti-C5 agent or a pharmaceutically acceptable salt thereof, wherein the subject has high-risk drusen, and wherein the progression of the ophthalmological disease, disorder, and/or condition is reduced, slowed, inhibited, prevented, improved, and/or reversed. In some of the above embodiments, at least one characteristic of drusen (e.g., number, extent, area, volume, shape, density, and reflectivity) is reduced, slowed, inhibited, prevented, improved and/or reversed.

In an embodiment, the subject has risk factors for the progression to iRORA and is intravitreally administered 2 mg per/eye of ARC1905 monthly.

In an embodiment, the subject has iRORA and is intravitreally administered 2 mg per/eye of ARC1905 monthly.

In an embodiment, the subject has nGA and is intravitreally administered 2 mg per/eye of ARC1905 monthly.

In an embodiment, the subject has intermediate AMD and is intravitreally administered 2 mg per/eye of ARC1905 monthly.

In an embodiment, the subject has cRORA and is intravitreally administered 2 mg per/eye of ARC1905 monthly.

In an embodiment, the subject has high-risk drusen and risk factors for iRORA and is intravitreally administered 2 mg per/eye of ARC1905 monthly.

In an embodiment, the subject has high-risk drusen and iRORA and is intravitreally administered 2 mg per/eye of ARC1905 monthly.

In an embodiment, the subject has high-risk drusen and nGA and is intravitreally administered 2 mg per/eye of ARC1905 monthly.

In an embodiment, the subject has high-risk drusen and intermediate AMD and is intravitreally administered 2 mg per/eye of ARC1905 monthly.

In an embodiment, the subject has high-risk drusen and cRORA and is intravitreally administered 2 mg per/eye of ARC1905 monthly.

In an embodiment, the subject has high-risk drusen and GA and is intravitreally administered 2 mg per/eye of ARC1905 monthly.

In an embodiment, the subject has high-risk drusen and AMD and is intravitreally administered 2 mg per/eye of ARC1905 monthly.

In an embodiment, the subject has high-risk drusen and dry AMD and is intravitreally administered 2 mg per/eye of ARC1905 monthly.

In an embodiment, the subject high-risk drusen and wet AMD and is intravitreally administered 2 mg per/eye of ARC1905 monthly.

### Other Compounds

In some embodiments, the methods described herein may further comprise administration of an anti-C5 agent or a pharmaceutically acceptable salt thereof described herein with a HTRA1 inhibitor compound, such as a HTRA1 inhibitor compound disclosed in U.S. Pat. Nos. 10,526,315, 10,730,832, and U.S. patent application Ser. No. 16/312,247, whose contents are incorporated herein by reference in their entirety.

The term “VEGF” refers to a vascular endothelial growth factor that induces angiogenesis or an angiogenic process. As used herein, the term “VEGF” may include the various subtypes of VEGF. Further, as used herein, the term “VEGF” may include VEGF-related angiogenic factors such as PIGF (placenta growth factor), VEGF-B, VEGF-C, VEGF-D and VEGF-E, which act through a cognate VEFG receptor (e.g., VEGFR) to induce angiogenesis or an angiogenic process. The term “VEGF” may include any member of the class of growth factors that binds to a VEGF receptor such as VEGFR-1 (Flt-1), VEGFR-2 (KDR/FIk-1), or VEGFR-3 (FLT-4). The term “VEGF” can be used to refer to a “VEGF” polypeptide or a “VEGF” encoding gene or nucleic acid.

The term “VEGF antagonist” refers to an agent that reduces, or inhibits, either partially or fully, the activity or production of a VEGF. In certain embodiments, the VEGF antagonist inhibits one or more of VEGF-A, VEGF-B, VEGF-C and VEGF-D. A VEGF antagonist can directly or indirectly reduce or inhibit the activity or production of a specific VEGF such as VEGFi65. Furthermore, “VEGF antagonists” may include agents that act on either a VEGF ligand or its cognate receptor so as to reduce or inhibit a VEGF-associated receptor signal. Examples of “VEGF antagonists” may include antisense molecules, ribozymes or RNAi that target a VEGF nucleic acid; anti-VEGF aptamers, anti-VEGF antibodies to VEGF itself or its receptor, or soluble VEGF receptor decoys that prevent binding of a VEGF to its cognate receptor; antisense molecules, ribozymes, or RNAi that target a cognate VEGF receptor (VEGFR) nucleic acid; anti-VEGFR aptamers or anti-VEGFR antibodies that bind to a cognate VEGFR receptor; and VEGFR tyrosine kinase inhibitors. In certain embodiments, the VEGF antagonist is a peptide, e.g., a peptide comprising three or more amino acid residues. In certain embodiments, the VEGF antagonist is a bicyclic peptide. In certain embodiments, the VEGF antagonist is a gene therapy, vector, or delivery vehicle delivering a plasmid, gene, or other genetic sequence capable of giving rise to a moiety that antagonizes VEGF.

### Example 1

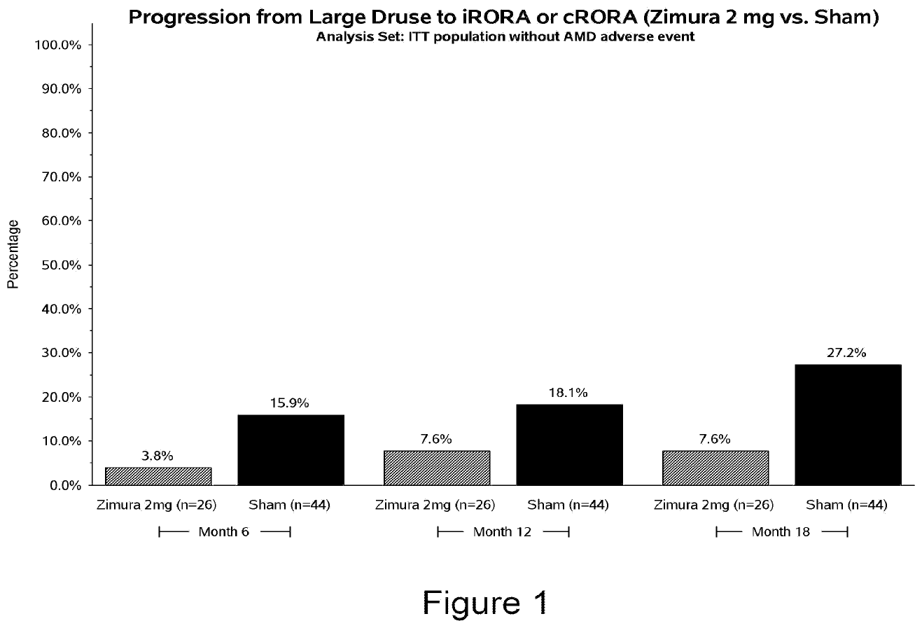

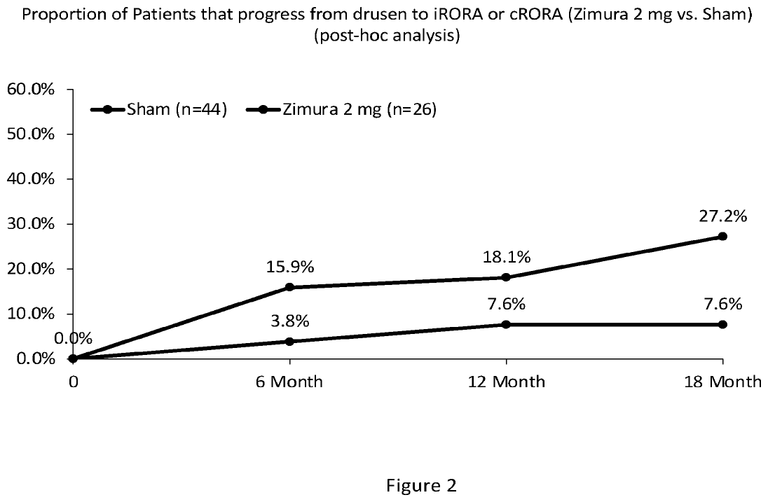

Subjects were administered 2 mg of the anti-C5 agent, ARC1905, or a sham and the progression from large drusen to iRORA or cRORA after administration at 6 months, 12 months, and 18 months was observed. As shown in FIGS. 1 and 2, after 6 months, 3.8% of the patients that were administered 2 mg of the anti-C5 agent progressed from large drusen to iRORA or cRORA, while 15.9% of the control group (i.e., “sham”) progressed from large drusen to iRORA or cRORA. Further, after 12 months, 7.6% of the patients that were administered 2 mg of the anti-C5 agent progressed from large drusen to iRORA or cRORA, while 18.1% of the control group progressed from large drusen to iRORA or cRORA. Lastly, after 18 months, 7.6% of the patients that were administered 2 mg of the anti-C5 agent progressed from large drusen to iRORA or cRORA, while 27.2% of the control group progressed from large drusen to iRORA or cRORA. As such, with regard to the progression of drusen to either iRORA or cRORA, a 19.6% reduction was observed, representing a 76% relative reduction in risk with 2 mg of the anti-C5 agent as compared to sham at 18 months. This effect was early and sustained with no additional patients progressing between M12 and M18. Further, these observations highlight the potential of preserving cells that are yet to be committed to atrophy, as they are with iRORA.

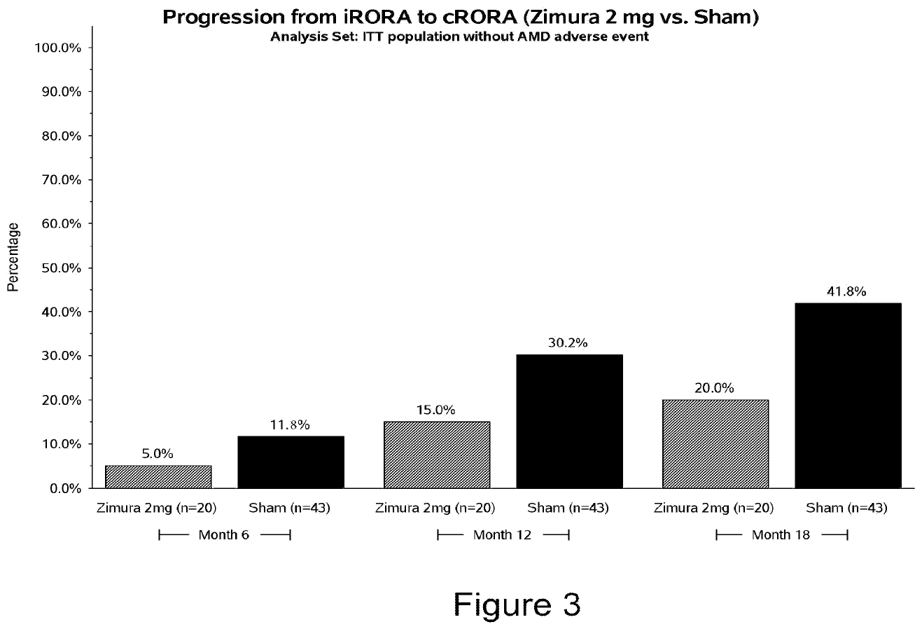

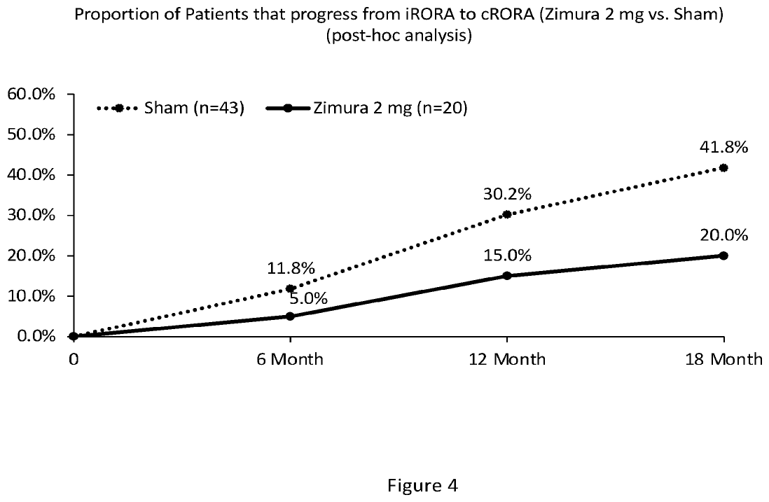

Progression from iRORA to cRORA after administration at 6 months, 12 months, and 18 months was also observed for subjects administered 2 mg of the anti-C5 agent, ARC1905, or a sham. As shown in FIGS. 3 and 4, after 6 months, 5.0% of the patients that were administered 2 mg of the anti-C5 agent progressed from iRORA to cRORA, while 11.8% of the control group progressed from iRORA to cRORA. Further, after 12 months, 15.0% of the patients that were administered 2 mg of the anti-C5 agent progressed from iRORA to cRORA, while 30.2% of the control group progressed from iRORA to cRORA. Lastly, after 18 months, 20.0% of the patients that were administered 2 mg of the anti-C5 agent progressed from iRORA to cRORA, while 41.8% of the control group progressed from iRORA to cRORA. As such, with regard to the progression of incomplete RPE and outer retinal atrophy to complete RPE and outer retinal atrophy, a reduction in the rate of progression by approximately 50% was observed with 2 mg of the anti-C5 agent as compared to sham. The effect was early and sustained, being observed at the month 6, month 12, and month 18 timepoints.

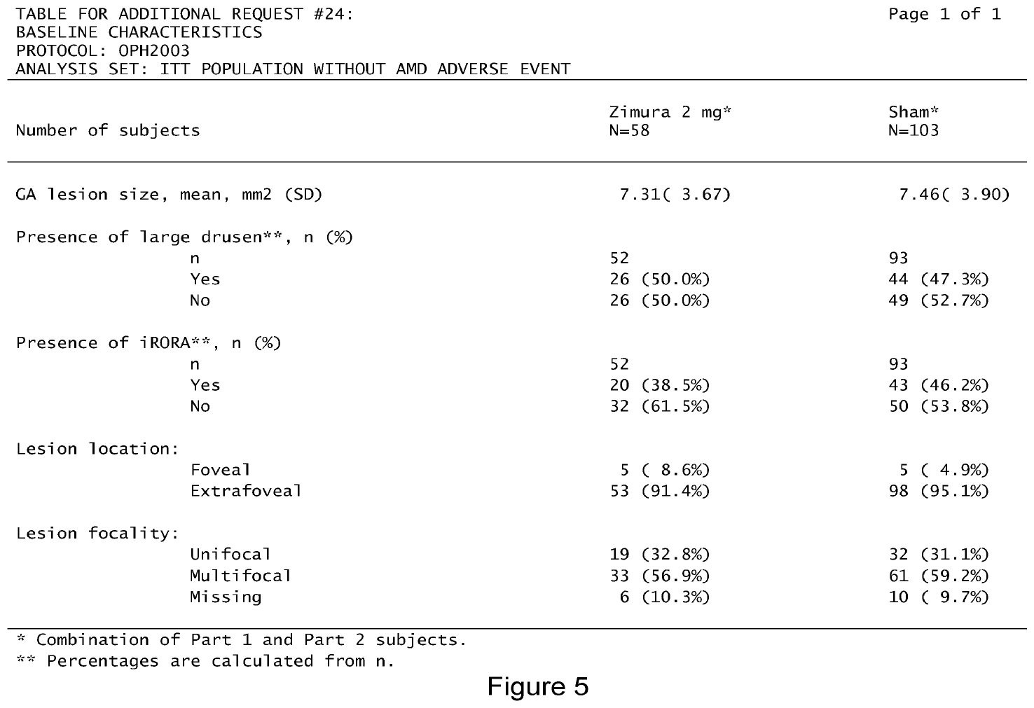

FIG. 5 compares baseline characteristics of GA lesion size, presence of large drusen, presence of iRORA, lesion location, and lesion focality for subjects administered 2 mg of the anti-C5 agent or sham. 50/of the patients that were administered 2 mg of the anti-C5 agent had large drusen, while only 47.3% of subjects administered the sham had large drusen. However, 38.5% of the patients that were administered 2 mg of the anti-C5 agent had iRORA, while 46.2% of the control group had iRORA.

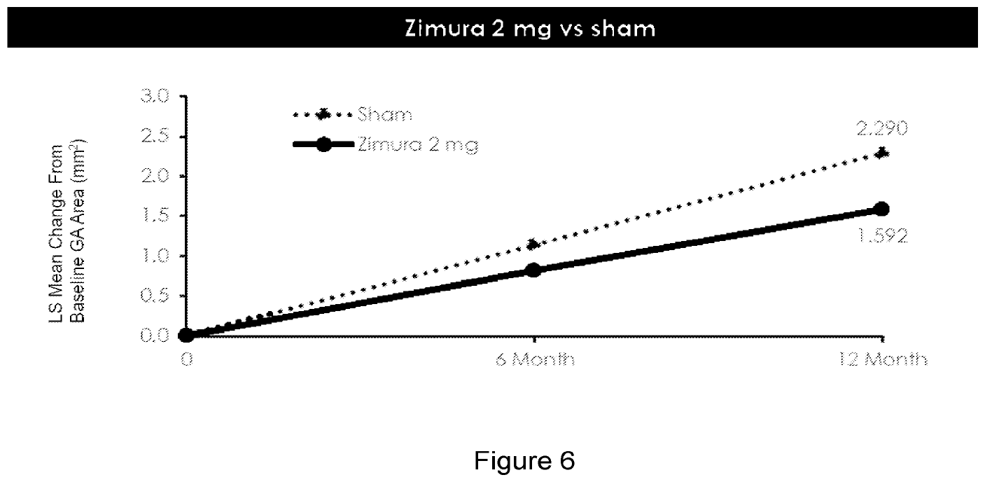

FIG. 6 compares the least-squares mean change from baseline GA area after administration of 2 mg of the anti-C5 agent versus a sham at 6 months and 12 months. In this regard, after 12 months, there was a 1.592 least-squares mean change from baseline GA in patients that were administered 2 mg of the anti-C5 agent, while there was a 2.290 least-squares mean change from baseline GA in sham.

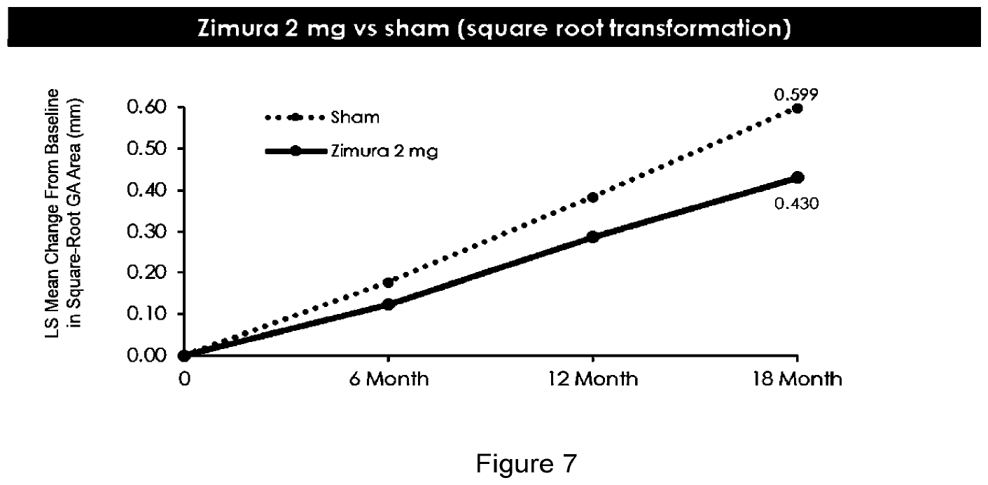

FIG. 7 compares the least-squares mean change from baseline in square-root GA area after administration of 2 mg of the anti-C5 agent versus a sham at 6 months, 12 months, and 18 months. In this regard, after 18 months, there was a 0.430 least-squares mean change from baseline GA in patients that were administered 2 mg of the anti-C5 agent, while there was a 0.599 least-squares mean change from baseline GA in sham.

This example demonstrates that administration of an anti-C5 agent is an effective therapy for slowing development of GA in late AMD and is beneficial if applied to an earlier disease state.

The foregoing description is given for clearness of understanding only, and no unnecessary limitations should be understood therefrom, as modifications within the scope of the invention may be apparent to those having ordinary skill in the art.

Throughout this specification and the claims which follow, unless the context requires otherwise, the word “comprise” and variations such as “comprises” and “comprising” will be understood to imply the inclusion of a stated integer or step or group of integers or steps, but not the exclusion of any other integer or step or group of integers or steps.

Throughout the specification, where compositions are described as including components or materials, it is contemplated that the compositions can also consist essentially of, or consist of, any combination of the recited components or materials, unless described otherwise. Likewise, where methods are described as including particular steps, it is contemplated that the methods can also consist essentially of, or consist of, any combination of the recited steps, unless described otherwise. The invention illustratively disclosed herein suitably may be practiced in the absence of any element or step which is not specifically disclosed herein.

The practice of a method disclosed herein, and individual steps thereof, can be performed manually and/or with the aid of or automation provided by electronic equipment. Although processes have been described with reference to particular embodiments, a person of ordinary skill in the art will readily appreciate that other ways of performing the acts associated with the methods may be used. For example, the order of various steps may be changed without departing from the scope or spirit of the method, unless described otherwise. In addition, some of the individual steps can be combined, omitted, or further subdivided into additional steps.

All patents, publications, and references cited herein are hereby fully incorporated by reference. In case of conflict between the present disclosure and incorporated patents, publications and references, the present disclosure should control.

What is claimed:

1. A method for treating an ophthalmological disease, disorder, and/or condition comprising intravitreally administering a pegylated anti-C5 agent at a dose of about 2 mg/eye to a subject in need thereof having: (i) high-risk drusen and (ii) an ophthalmological disease, disorder, and/or condition selected from the group consisting of risk factors for the progression to iRORA, iRORA, nGA, cRORA, GA, AMD, intermediate AMD, dry AMD, and wet AMD,
wherein the pegylated anti-C5 agent is an Aptamer, wherein the Aptamer=fCmGfCfCGfCmGmGfUfCfUfCmAmGmGfCGfCfUmGmAmGfJfCfUmGmAmGfUfUfUAfCf CfUmGfCmG-3T (SEQ ID NO: 1), wherein fC and fU=2′ fluoro nucleotides, mG and mA=2′-OMe nucleotides, all other nucleotides are 2′-OH, and 3T indicates an inverted deoxythymidine. 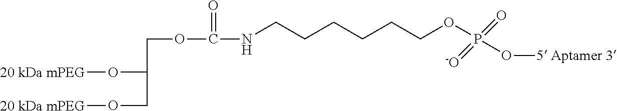
or a salt thereof.

3. The method of claim 1, wherein the subject has intermediate AMD.

4. The method of claim 1, wherein the subject has iRORA.

5. The method of claim 1, wherein the subject has cRORA

6. The method of claim 1, wherein the subject has nGA.

7. The method of claim 1, wherein the subject has GA.

8. A method for treating an ophthalmological disease, disorder, and/or condition selected from the group consisting of risk factors for the progression to iRORA, iRORA, nGA, cRORA, GA, AMD, intermediate AMD, dry AMD, and wet AMD, comprising intravitreally administering a pegylated anti-C5 agent at a dose of about 2 mg/eye to a subject in need thereof, wherein the subject has high-risk drusen,
wherein the pegylated anti-C5 agent is an Aptamer, wherein the Aptamer=fCmGfCfCGfCmGmGfUfCfUfCmAmGmGfCGfCfUmGmAmGfUfCfUmGmAmGfUfJfUAfCf CfUmGfCmG-3T (SEQ ID NO: 1), wherein fC and fU=2′ fluoro nucleotides, mG and mA=2′-OMe nucleotides, all other nucleotides are 2′-OH, and 3T indicates an inverted deoxythymidine. 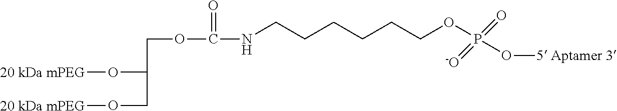
or a salt thereof.

10. The method of claim 8, wherein the subject has intermediate AMD.

11. The method of claim 8, wherein the subject has iRORA.

12. The method of claim 8, wherein the subject has cRORA

13. The method of claim 8, wherein the subject has nGA.

14. A method for treating an ophthalmological disease, disorder, and/or condition selected from the group consisting of risk factors for the progression to iRORA, iRORA, nGA, intermediate AMD, and cRORA, comprising intravitreally administering a pegylated anti-C5 agent at a dose of about 2 mg/eye to a subject in need thereof,
wherein the pegylated anti-C5 agent is an Aptamer, wherein the Aptamer=fCmGfCfCGfCmGmGfUfCfUfCmAmGmGfCGfCfUmGmAmGfUfCfUmGmAmGfUfUfUAfCf CfUmGfCmG-3T (SEQ ID NO: 1), wherein fC and fU=2′ fluoro nucleotides, mG and mA=2′-OMe nucleotides, all other nucleotides are 2′-OH, and 3T indicates an inverted deoxythymidine. 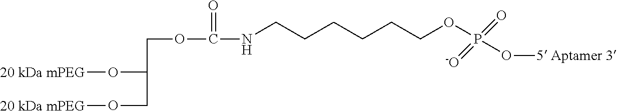
or a salt thereof.

16. The method of claim 14, wherein the subject has intermediate AMD.

17. The method of claim 14, wherein the subject has iRORA.

18. The method of claim 14, wherein the subject has risk factors for the progression to iRORA.

19. The method of claim 14, wherein the subject has nGA.

20. The method of claim 14, wherein the subject has cRORA.
